(19) United States
(12) Patent Application Publication
(10) Pub. No.: US 2025/0010025 A1
(43) Pub. Date: Jan. 9, 2025

(54) CATHETER ASSEMBLY

(71) Applicant: ConvaTec Limited, Flintshire (GB)

(72) Inventors: Oliver Pfleger, Spital (GB); Lukasz Natkaniec, Munchen (DE); Phillip Emrich, Munchen (DE)

(21) Appl. No.: 18/891,582

(22) Filed: Sep. 20, 2024

Related U.S. Application Data
(63) Continuation of application No. PCT/GB2023/050693, filed on Mar. 20, 2023.
(60) Provisional application No. 63/349,641, filed on Jun. 7, 2022.
(60) Provisional application No. 63/394,710, filed on Aug. 3, 2022.

(30) Foreign Application Priority Data
Mar. 21, 2022 (GB) GB2203882.2
Aug. 12, 2022 (GB) 2211843.4

(51) Int. Cl. A61M 25/00

(52) U.S. Cl. CPC A61M 25/0017; A61M 25/0009; A61M 25/002; A61M 2205/583; A61M 2207/10; A61M 2209/00

(57) ABSTRACT

A catheter assembly that has a fluid collection bag and a catheter. The fluid collection bag has two panels, a peripheral bond between the two panels, and an inlet to receive fluid from the catheter. The two panels are configured to form a base of the fluid collection bag distal from the inlet; and the fluid collection bag is configured to stand up on the base. The fluid collection bag may have a front, rear and base panels. The base panel may form the base of the bag. The fluid collection bag may have two side panels. The front and rear panels may be joined to opposite sides of each side panel. The base panel may be joined to one side of each side panel. The catheter may be a male urinary catheter.

### CROSS-REFERENCE TO RELATED DISCLOSURE

The present disclosure is a Continuation of International Application No. PCT/GB2023/050693 filed Mar. 20, 2023 and claims the benefit of GB2203882.2 filed Mar. 21, 2022; GB2211843.4 filed Aug. 12, 2022; U.S. 63/349,641 filed Jun. 7, 2022; and U.S. 63/394,710 filed Aug. 3, 2022, the disclosures of which being incorporated herein in entirety by reference.

### TECHNICAL FIELD OF THE INVENTION

The present invention relates to catheter assemblies. In particular the invention concerns packaged urinary catheter assemblies, and most particularly, but not exclusively packaged intermittent male urinary catheter assemblies, especially “closed” catheter assemblies.

### BACKGROUND TO THE INVENTION

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows them to enjoy a relatively normal lifestyle.

To reduce the risk of damage to the urinary tract and improve comfort while using the catheter, it is also important they are activated before use for example through wetting of the catheter by a wetting fluid. However, once wetted and/or used a catheter can be slippery and difficult to handle. This can make handling and re-packaging of the catheter after use difficult and unhygienic, as the catheter may inadvertently contact the user's hands, clothing or personal items. Existing intermittent catheters disclose continuous handling sleeves that cover the entire length of the catheter. While these provide some assistance to handling, the sleeve can make the catheter more difficult to wet as wetting fluid may not be able to easily reach all parts of the catheter.

To allow self-catheterisation as required, closed catheter assemblies collect and retain urine using a fluid collection bag, which removes the need for urine to pass directly into a toilet. To provide a fluid collection bag that is discreet prior to use but also able to store a suitable volume of liquid, fluid collection bags can be substantially 2D prior to use, consisting of two panels joined at their periphery. However, this can cause problems for the user as they must support or hold the fluid collection bag to ensure that it fills during use.

Once used, a fluid collection bag is heavy and bulky as it is filled with liquid. To allow the bag to be carried and disposed of more discretely and easily, existing bags may be provided with a drain to allow them to be emptied into a toilet. However, existing drains can either leak or are difficult to open which potentially results in inadvertent or uncontrolled release of liquid from the bag. In addition, the drain can add to the bulk of the catheter assembly which makes it less discreet and consequently less desirable for the user.

To provide discrete and portable catheter assemblies, packaging may be provided to contain the catheter and any other equipment such as wetting reservoirs or sachets and fluid collection bags before and/or after use. However, where liquid is contained within a sachet, reservoir or bag within the packaging, any release (intentional or otherwise) of this may cause liquid to accidentally leak out onto parts of the catheter assembly that the user has to touch-such as a handling element, or onto the packaging causing problems for the user.

After use, the catheter can also cause problems for the user as it is dirty having just been inside the body and an inconvenient shape to be packaged/stowed discreetly as it is long and thin and resiliently biased into a straight configuration. This can mean that the user must carry additional bags or other equipment to store the catheter after use if it is not possible for them to dispose of it immediately. Where the catheter is attached to a fluid collection bag, any storage bags must also be able to contain the fluid collection bag which may also be full of liquid. This further increases the challenges of storing a used catheter assembly until disposal is possible.

Many existing packaged closed catheter assemblies contain a fluid reservoir or sachet together with a fluid collection bag and catheter all contained within a pouch. Prior to use, the fluid reservoir or sachet is burst to release wetting fluid to activate the catheter surface and render it lubricious. In releasing the wetting fluid, the fluid collection bag may also become wet, which makes handling and re-packaging of the bag difficult, in particular for users with limited dexterity. In addition, if a significant portion of wetting fluid contacts, and gets stuck to the fluid collection bag, there may not be sufficient wetting fluid to activate the whole catheter surface. As such, the lack of sufficient lubrication of the catheter surface can lead to discomfort and injury during use.

It is an object of embodiments of the present invention to at least partially overcome or alleviate the above problems and/or to provide an improved packaged catheter assembly.

### SUMMARY OF THE INVENTION

In broad terms, the present invention concerns a catheter assembly comprising a catheter. In addition, the present invention also concerns a packaged catheter assembly comprising a pouch and the catheter assembly, wherein the pouch is configured to contain the catheter assembly within it. The pouch may be configured to contain within it any one or more of: a catheter; a fluid reservoir; a fluid collection bag; and a sleeve, any of which may be as defined in the statements of invention below.

In a broad aspect, there is provided a catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter; and a second configuration in which the valve assembly permits fluid flow in a direction from the fluid collection bag towards the catheter. Preferably, in the first configuration the valve assembly permits fluid flow in a direction from the catheter towards the fluid collection bag. In the second configuration, the valve assembly may inhibit fluid flow in a direction from the catheter towards the fluid collection bag.

Accordingly, a first aspect of the present invention provides a catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag.

Advantageously, the two configurations of the valve assembly allow the user to efficiently fill the fluid collection bag and store fluid within it, as well as drain the bag in a controlled manner without the need to tear or open the bag. In addition, the valve assembly removes the need for a separate inlet and outlet in the fluid collection bag as a single opening in the fluid collection bag can operate as both the inlet and outlet, this reduces the manufacturing cost and complexity of the fluid collection bag. In addition, it makes the bag simpler to use and more robust.

The catheter may be a urinary catheter. The catheter may be a male urinary catheter. The catheter may be a female urinary catheter. The catheter may be an intermittent catheter. In one embodiment, the catheter is an intermittent male urinary catheter. Thus, the features of the present invention allow intermittent male urinary catheters to be used more easily and effectively.

The catheter may be formed of a material of the group comprising: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, and water disintegrable or enzymatically hydrolysable material, or combinations, blends or co-polymers of any of the above materials.

The water disintegrable or enzymatically hydrolysable material may comprise a material of the group comprising: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

Preferably, the intermittent catheter is formed of a polyolefin material, in particular polyethylene and/or polypropylene.

Preferably, the intermittent catheter is formed of a thermoplastic elastomeric material.

The valve assembly may be provided in the first configuration. For example, the catheter assembly may be provided in the first configuration in a sealed pouch. The valve assembly may be configured to move from the first configuration to the second configuration. The valve assembly may be configured to move from the second configuration to the first configuration. The valve assembly may be configured to move reversibly between the first and second configurations. Thus, the valve assembly is reversible between the first and second configurations which allows the user to effectively re-seal the fluid collection bag if necessary. This can help prevent further leaks after draining the bag which can be common in bags where an outlet is opened irreversibly (for example by tearing the bag) and fluid lining the inside of the fluid collection bag slowly collects and flows out.

The valve assembly may be configured to resist movement from the second configuration to the first configuration. The valve assembly may be configured to resist movement from the first configuration to the second configuration. The valve assembly may remain in the first or second configuration. Thus, the valve assembly may not be reversibly switched between the first and second configurations as once in a configuration it resists movement back to the other configuration. This can help reduce the risk of inadvertently changing the configuration multiple times when only one change of configuration is required.

The valve assembly may comprise two passageways: a proximal passageway and a distal passageway. The proximal passageway may be configured to provide a fluid communication between the valve assembly and the catheter. The distal passageway may be configured to provide a fluid connection between the valve assembly and the fluid collection bag. The valve assembly may comprise a valve housing. The proximal and distal passageways may connect to opposite sides of the valve housing. The valve housing may be any suitable shape such as cuboid, spherical, cylindrical or irregularly shaped, preferably it is cylindrical. The proximal and distal passageways may connect to a side wall of the valve housing. The valve housing may have a diameter (or width) that is at least 1 cm, 1.5 cm, 2 cm or 3 cm. The valve housing may have a diameter that is no more than 4 cm, 3 cm, 2 cm or 1.5 cm. Preferably, the valve housing has a diameter of 3 cm. The valve housing may have an aspect ratio (length:width/diameter) of at least 1:1, 2:1 or 3:1. The valve housing may have an aspect ratio (length:width/diameter) of no more than 3:1, 2:1 or 1:1. Preferably, the valve housing has an aspect ratio of (length:width/diameter) 2:1. Thus, the valve assembly can be conveniently be placed in-line with the catheter and fluid collection bag via the passageways.

Each passageway may extend away from the valve housing by a distance that is no more than twice the diameter/width of the valve housing, and preferably a distance that is no more than the diameter/width of the valve housing. Thus, the valve assembly itself remains compact and the passageways are less likely to be damaged by bending forces experienced during use/manipulation of the catheter and fluid collection bag.

Each passageway may be tubular. Each passageway may have an internal diameter that is the same size or greater than an internal diameter of the catheter. Thus, the passageways have a lower fluid resistance than the catheter and do not unnecessarily inhibit the speed of fluid flow between the catheter and fluid collection bag.

The valve assembly may comprise a user operable control. The user operable control may be configured to move the valve assembly from the first configuration to the second configuration. The user operable control may be configured to move the valve assembly from the second configuration to the first configuration. The user operable control may be configured to reversibly move the valve assembly between the first and second configurations. Alternatively, the user operably control may not be reversible and may irreversibly switch the valve assembly from the first to the second configuration. The user operable control may comprise any suitable user actuable input such as a switch, button, lever, slider, knob. Preferably, the user operable control comprises a button. The button may be any suitable shape or size but is preferably the same shape as one side or face of the valve housing. The button preferably covers a majority of a side or face of the valve housing. Where the valve housing is cylindrical, the button is preferably cylindrical. One end of the valve housing may be an open end. The button may fill the open end of the valve housing. The button may be configured to move axially with respect to the (cylindrical) valve housing. The button may have an extended configuration. The extended configuration of the button may correspond to the first configuration of the valve assembly. In the extended configuration the button may extend beyond the open end of the valve housing. The button may have a depressed configuration. The depressed configuration of the button may correspond to the second configuration of the valve assembly. In the depressed configuration the button may be closer to the open end of the valve housing than when it is in the extended configuration. Preferably in the depressed configuration the button is flush with the open end of the valve housing. The valve assembly is therefore easy to use as the user can simply press down anywhere on one side of the valve assembly to operate the valve assembly, this is particularly useful where a user has reduced manual dexterity.

The user operable control may be biased to move the valve assembly to the first configuration. The user operable control may comprise a biasing element, for example a spring or other material/object that can be resiliently deformed by a user's hand or grip strength. The biasing element may be configured to bias the user operable control to move the valve assembly to the first configuration. The user operable control may be biased to keep the valve assembly in the first configuration. When the valve assembly is in the first configuration, the biasing element may be configured to bias the user operable control to keep the valve assembly to the first configuration.

The user operable control may be biased to move the valve assembly to the second configuration. The biasing element may be configured to bias the user operable control to move the valve assembly to the second configuration. The user operable control may be biased to keep the valve assembly in the second configuration. When the valve assembly is in the second configuration, the biasing element may be configured to bias the user operable control to keep the valve assembly to the second configuration. Thus, the user operable control helps to ensure that inadvertent switching between the first and second configurations is avoided.

The user operable control may comprise a switching point. The biasing element may be configured to reverse the bias of the user operable control when the user operable control is moved to and/or past the switching point. When the valve assembly is in the first configuration, the biasing element may be configured to bias the user operable control to move the valve assembly to the second configuration when the user operable control is moved to and/or past the switching point. When the valve assembly is in the second configuration, the biasing element may be configured to bias the user operable control to move the valve assembly to the first configuration when the user operable control is moved to and/or past the switching point. The switching point may correspond to the depressed configuration of the button. The switching point may correspond to a position in which the button is depressed further than the depressed configuration. Thus, the switching point ensures the valve assembly is only switched between the first and second configurations when the user intends which reduces the risk of leaks through the valve assembly.

The valve assembly may be configured to provide feedback as it switches between the first and second configurations, for example an audible/tactile output such as a “click” and/or a change of visual appearance. An audible and/or tactile output may be provided by any suitable means such as via a protrusion on the inside of the valve housing that overrides a detent on the button as the button moves with respect to the valve housing.

The valve assembly may be configured to indicate if it is in the first or second configuration. The user operable control may be configured to change the appearance of at least part of the valve assembly as the valve assembly is moved between the first and second configurations. The button may comprise a side wall that is a different colour or shade of colour from the valve housing. The side wall of the button may be visible when the button is in the extended configuration/the valve assembly is in the first configuration. The side wall of the button may be hidden by the valve housing when the button is in the depressed configuration/the valve assembly is in the second configuration. Thus, button provides a visual reference to the state of the valve assembly.

The valve housing may comprise a window. The window may be located in a side wall of the valve housing. The window may be configured to change appearance depending on if the valve assembly is in the first configuration or second configuration. The window may be located equidistance from each of the passageways. The window may be elongate. The window may comprise a length in a direction parallel to the axis of the (cylindrical) valve housing. The window may have a length that is approximately half the diameter/width of the valve housing. The window may have an aspect ratio (length:width) or 1:1, 2:1, 3:1 or 4:1, preferably 2:1. The window may be any suitable shape, such as circular, semi-circular, triangular, rectangular, or irregularly shaped, preferably, the window is stadium-shaped, that is rectangular semicircles replacing two opposite sides. The button may comprise a side wall with two regions which are visually distinct from one another, for example they may be a different colour, a different shade of colour and/or comprise different markings on them. One of the regions may correspond to the position of the window when the button is in the extended configuration and the other region may correspond to the position of the window when the button is in the depressed configuration. One region may be the same colour as the side wall of the button and the other may be a different colour/shade of colour as the side wall of the button. Alternatively, both regions may be a different colour or shade of colour from the side wall of the button. Thus, a window can provide a simple visual reference to the state of the valve assembly as a specific region of the button is visible through the window depending on the state of the button and therefore the valve assembly.

A top end of the button, that is the end that is outside the valve housing, may comprise an activation marker. The activation marker may comprise a visual indication to signal to the user how to use the button, for example it may comprise the word “PUSH” written on the button. The side wall of the button may also comprise an activation marker, for example an arrow pointing in the direction the button should be depressed. Thus, the operation of the button is intuitive for the use.

The valve assembly may comprise a valve body inside the valve housing. The valve body may be fluidly connected to the catheter (e.g. via the proximal passageway). The valve body may be fluidly connected to the fluid collection bag (e.g. via the distal passageway). The valve body may be connected inline between the catheter and fluid collection bag (e.g. via the proximal and distal passageways). The valve body may be configured to inhibit fluid flow in a direction from the fluid collection bag towards the catheter when the valve assembly is in the first configuration. The valve body may be configured to permit fluid flow in a direction from the catheter towards the fluid collection bag when the valve assembly is in the first configuration. The valve body may be configured to permit fluid flow in a direction from the fluid collection bag towards the catheter when the valve assembly is in the second configuration. The valve body may be configured to inhibit fluid flow in a direction from the catheter towards the fluid collection bag when the valve assembly is in the second configuration. The valve body may be coupled to the user operable control. Thus, the valve body enables the valve assembly to function as required.

The valve body may be configured to inhibit fluid flow in a direction from the distal passageway towards the proximal passageway when the valve assembly is in the first configuration. The valve body may be configured to permit fluid flow in a direction from the proximal passageway towards the distal passageway when the valve assembly is in the first configuration. The valve body may be configured to permit fluid flow in a direction from the distal passageway towards the proximal passageway when the valve assembly is in the second configuration. The valve body may be configured to inhibit fluid flow in a direction from the proximal passageway towards the distal passageway when the valve assembly is in the second configuration.

The valve body may be any suitable fluidic system to enable the valve assembly to operate as described above. The valve body may comprise a first check valve. The first check valve may be connected to the catheter (e.g. via the proximal passageway). The first check valve may be connected to the fluid collection bag (e.g. via the distal passageway). The first check valve may be configured to inhibit fluid flow in a direction from the fluid collection bag towards the catheter. The first check valve may be configured to permit fluid flow in a direction from the catheter towards the fluid collection bag. The valve body may be configured to connect the first check valve to the catheter and fluid collection bag when the valve assembly is in the first configuration. The valve body may be configured to disconnect the first check valve from the catheter and fluid collection bag when the valve assembly is in the second configuration. The valve body may be configured to connect the catheter and fluid collection bag by a tube (or a second check valve with the opposite polarity to the first check valve) when the valve assembly is in the second configuration. Thus, a simple check valve and tube may be used to operate the valve assembly. In an alternative, the first check valve could be operable to reverse its polarity in the second configuration. For example, a user operable control such as a lever or handle, could rotate the first check valve to reverse its polarity.

The valve body may be configured to move (e.g. axially) within the valve housing. The valve body may be configured to move the first check valve (e.g. axially) within the valve housing to connect and disconnect it from the proximal and distal passageways. The first check valve may be coupled to (and may move with) the button. The first check valve and tube (or second check valve) may be stacked (e.g. axially) within the valve housing. The valve body and user operable control/button may be integrally formed. As such, simple operation of the button allows the valve assembly to be operated with minimal additional components, of course, the first check valve, tube, and second check valve could be replaced with other fluidic components that operate in a similar manner to achieve the same result/benefits as the example given above.

The valve assembly may be directly connected to the fluid collection bag, for example the fluid collection bag may be directly connected to the distal passageway. Thus, the catheter assembly can form a compact unit and the operation of the valve assembly is more intuitive for the user as it is close to the bag.

The distal passageway may be connected to an access opening of the fluid collection bag. The access opening may be configured to allow fluid access to the inside of the fluid collection bag, thus the access opening may function as both an inlet and outlet of the bag. As such, any feature of an inlet or outlet described herein may equally apply to the access opening. The access opening may be formed of a flexible material. This is useful to allow the valve assembly to be moved with respect to the bag and furthermore to allow for more compact packaging of the fluid collection bag and valve assembly.

The valve assembly may be indirectly connected to the fluid collection bag, for example via an intervening tube or other fluidic component. This can allow the valve assembly to be more conveniently accessed by the user in some circumstances.

The catheter assembly may comprise a sleeve configured to enclose the catheter along the length of the catheter from the proximal end to the distal end. The sleeve may be attached to the valve assembly, preferably the sleeve is attached to the proximal passageway. The catheter and/or sleeve may be configured to deliver fluid to the valve assembly/proximal passageway. The catheter and/or sleeve may be configured to receive fluid from the valve assembly/proximal passageway. The sleeve may be configured to receive fluid from the valve assembly during draining of the fluid collection bag. The valve assembly may be directly connected to the sleeve, for example the sleeve may be directly connected to the proximal passageway. Thus, the sleeve can be used to ensure a simple and effective fluid connection between the catheter and the valve assembly.

The fluid collection bag may comprise a panel. The fluid collection bag may comprise an inlet (for example the access opening may function as an inlet). The fluid collection bag may comprise a base. The fluid collection bag may be configured to stand up on the base. Consequently, in a broad aspect, there is provided a catheter assembly comprising a fluid collection bag and a catheter; the fluid collection bag comprising a panel, an inlet to receive fluid from the catheter, and a base, wherein the fluid collection bag is configured to stand up on the base. The fluid collection bag may comprise two panels. The fluid collection bag may comprise a peripheral bond between the two panels. The two panels may be configured to form the base. The base may be distal from the inlet.

Accordingly, a second aspect of the present invention provides a catheter assembly comprising a fluid collection bag and a catheter; the fluid collection bag comprising at least two panels, a peripheral bond between the two panels, and an inlet to receive fluid from the catheter, wherein: the two panels are configured to form a base of the fluid collection bag distal from the inlet; and the fluid collection bag is configured to stand up on the base.

Advantageously, the fluid collection bag is free-standing and is more convenient to use as it does not need to be hung or held during filling. As it is free-standing, with the inlet distal the base, the bag also fills more efficiently with liquid and can ensure that more of the bag is filled with liquid. This means the fluid collection bag can have a smaller form factor which is cheaper, lighter and more discreet for the user.

The distal end of the catheter may comprise a funnel. The funnel may be formed of a relatively rigid material compared to the rest of the catheter and/or fluid collection bag, for example high density poly-ethylene (HDPE). The funnel may have a truncated conical shape. The funnel may be joined to the distal end of the catheter at the narrow end of the funnel. The inlet may be configured to prevent the catheter from being (completely) removed from the fluid collection bag, for example separation of the catheter from the fluid collection bag. The inlet may engage with a part of the catheter, preferably the distal end and/or funnel. Thus, fluid can be securely and easily transferred from the catheter into the fluid collection bag and the risk of leaks associated with the separation of the catheter from the fluid collection bag is minimised (through engagement of the inlet and catheter).

The fluid collection bag may be any suitable shape or size, for example rectangular, circular, elliptical, cuboid, spherical, etc. The fluid collection bag may comprise a front panel and a rear panel. A peripheral bond may join at least part of the periphery of the front panel and rear panel to form the fluid collection bag. The peripheral bond may define a base, two lateral edges and/or an upper edge of the fluid collection bag. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag.

The fluid collection bag may have a width between the left lateral edge and right lateral edge. The width of the fluid collection bag may be at least 20, 25, 30 or 35 cm. The bag may have a width no more than 40, 35, 30, or 25 cm. Preferably, the width is between 30 and 35 cm, most preferably between 33 and 35 cm. The base may define a bottom of the bag. The upper edge may define a top of the bag. The length/height of the bag may be defined from the base to the upper edge. The height of the bag may be at least 15, 20, 25, 30, 35 or 40 cm. The height of the bag may be no more than 45, 40, 35, 30, 25, or 20 cm. The height may be between 20 and 40 cm, 25 and 40, 20 and 30 cm, 30 and 35 cm or most preferably between 33 and 35 cm, or 28 cm. Thus, preferably, the bag is square. The bag may be configured to hold at least 500, 700, or 1000 ml of fluid, and may hold no more than 1000 ml. For example, it may hold at least 700 ml of fluid.

The fluid collection bag may comprise a side panel, for example two side panels. The side panels may be configured to allow the front and rear panels to at least partially separate. Thus, the side panels allow the fluid collection bag to hold a greater volume of liquid.

Accordingly in one embodiment there is provided a catheter assembly comprising a fluid collection bag and a catheter; the fluid collection bag comprising two panels, a peripheral bond between the two panels, and an inlet to receive fluid from the catheter, wherein: the two panels are configured to form a base of the fluid collection bag distal from the inlet; and the fluid collection bag is configured to stand up on the base wherein the fluid collection bag comprises a front panel, a rear panel, two side panels and a base panel, and the front and rear panels are joined to opposite sides of the base panel, the front and rear panels are joined to opposite sides of each side panel, and the base panel is joined to one side of each side panel, wherein the base panel is configured to form a base of the fluid collection bag.

Each side panel may be any suitable shape, for example lens shaped, rectangular or triangular. Preferably, the side panel is triangular, for example an isosceles triangle. Each side panel may have a height that is equal to the height of the fluid collection bag. The front panel may be joined to each side panel. The rear panel may be joined to each side panel. The front and rear panels may be joined to opposite sides of each side panel. Each side panel may have two equal length sides. The two equal length sides of a side panel may be bonded to the front and rear panels respectively. One side panel may be on a side of the fluid collection bag corresponding to the left lateral edge, and the other may be on the side corresponding to the right lateral edge. The side panels may be configured to form a base of the fluid collection bag. The side panels may be configured to allow the front and rear panels to separate further at the base of the fluid collection bag than at the upper edge of the fluid collection bag. The side panels may be configured to prevent separation of the fluid collection bag along the upper edge of the fluid collection bag. For example, the front panel and the rear panel may be directly joined to each other along the upper edge of the fluid collection bag. The two side panels may be configured to allow the front and rear panels to separate along the left lateral edge and/or right lateral edge of the fluid collection bag. The fluid collection bag may be wedge shaped. Thus, the side panels efficiently create additional volume within the bag and allow it to form a free-standing wedge shape.

The fluid collection bag may comprise a base panel. The base panel may span between the front panel and rear panel along the base of the peripheral bond. The base panel may be configured to form a base of the fluid collection bag. That is to say the base of the fluid collection bag may be formed by the front panel, the rear panel and the base panel, optionally also the two side panels. The base panel and side panels may be integrally formed. The base panel may be configured to allow the front panel and rear panel to separate to form the base of the fluid collection bag. The front panel may be joined to the base panel. The rear panel may be joined to the base panel. The front and rear panels may be joined to opposite sides of the base panel. The base may be formed by two flanges; the rear panel joined to the base panel (and/or side panels) may form one flange and the front panel joined to the base panel (and/or side panels) may form the other flange. Each flange may extend from one side of the upper edge of the peripheral bond to the other around the fluid collection bag, that is down a lateral edge (e.g. right lateral edge) along the base and back up the other lateral edge (e.g. left lateral edge). Consequently, the front panel and rear panel may be (directly) joined by the peripheral bond (only) along an upper edge of the fluid collection bag. Thus, the base panel and side panels may be used to form a base of the bag and allow it to be free-standing.

The base panel may be elongate. The base panel may comprise two elongate edges corresponding to the edges of the front and rear panels. The base panel may comprise two side edges corresponding to the edges of the two side panels. The base panel may be rectangular. The two flanges may extend from the two elongate edges. The base panel may comprise two curved edges. The base panel may be lens shaped. The front and rear panel may be joined at the two respective curved edges. The two flanges may extend from the two curved edges. Thus, the base panel is efficiently attached to the fluid collection bag and does not interfere with the lateral edges of the bag.

The base panel (and/or each side panel) may comprise a stowed configuration. In the stowed configuration, the base panel (and/or each side panel) may be configured to reduce the internal volume of the fluid collection bag. The base panel (and/or each side panel) may be folded in the stowed configuration. The base panel (and/or each side panel) may be folded between the front and rear panels. The base panel (and/or each side panel) may be configured to fold to allow the front and rear panels to come together. Thus, the base panel and/or each side panel may be folded to allow the fluid collection bag to reduce in size which assists with storage and transport of the bag making it lighter and more discreet for the user.

The base panel (and/or each side panel) may be configured to automatically unfold. The base panel (and/or each side panel) may be configured to unfold as liquid enters the fluid collection bag. Thus, the fluid collection bag can be automatically deployed into a standing configuration as liquid enters the bag, for example under the weight of liquid entering/inside the bag. This makes the bag easier to use as the user does not need to manually unfold it.

The fluid collection bag may comprise a flexible plastics material. For example, polypropylene (PP), polyethylene terephthalate (PET), low density polyethylene (LDPE), metalized polyester (MET PET) orientated polypropylene (OPP) or polyvinyl chloride (PVC). The fluid collection bag may be formed of an opaque material. At least part of the fluid collection bag may be transparent or translucent. The front panel of the fluid collection bag may be transparent or translucent. Thus, the fluid collection bag can conform to the shape of the catheter assembly, is convenient to carry and also provides a visual reference to the contents of the bag before, during and after use which makes it easier to use.

The fluid collection bag may comprise a fill level indicator. The fill level indicator may be configured to allow the user to visualise fluid within the bag. Where the fluid collection bag is formed of an opaque material, the fill level indicator may comprise a region of the fluid collection bag, such as part of the front panel, that is formed of a transparent or translucent material. The fill level indicator may be any suitable shape or size such as circular, semi-circular, triangular, rectangular or irregularly shaped, preferably it is rectangular. The fill level indicator may have a height that spans a majority of the height of the fluid collection bag, for example at least 50%, 60%, 70%, 80%, or 90% of the height of the fluid collection bag. Preferably, the fill level indicator has a height of over 90% of the height of the fluid collection bag. The fill level indicator may have a width that spans a minority of the width of the fluid collection bag, for example, no more than 50%, 40%, 30% or 20% of the width of the fluid collection bag. The fill level indicator may have a width of at least 10%, 20%, 30%, or 40% of the width of the fluid collection bag. Preferably, the fill level indicator has a width of 30-50%, or 35-40% of the width of the fluid collection bag, for example, 37%.

The fill level indicator may comprise fill markers. The fill markers may allow the user to measure the volume of fluid contained within the fluid collection bag. The fill markers may be regularly spaced or irregularly spaced. The fill markers may correspond to an absolute volume of liquid contained within the bag. The fill markers may correspond to a proportion of the volume of the bag which is filled with fluid. The fill markers may indicate when a safe fill level of the bag has been reached. The fill level indicator may be positioned in a transparent or translucent region of the bag. Thus, the fill level indicator ensures the bag is not overfilled.

The peripheral bond may comprise any one or more of: a weld; mechanical seal; heat seal; pressure seal; adhesive; solvent bond; ultraviolet bond; ultrasonic weld; laser weld; impulse weld; or friction weld. The peripheral bond is generally permanent but in some embodiments may be re-sealable, for example through a resealable adhesive, an additional adhesive that is activated prior or during resealing, or a hook and loop/hook and hook fastener. The peripheral bond may provide a water-tight, and preferably sterile, seal. Thus the peripheral bong can be easily constructed to suit the specific needs of the catheter assembly and ensure the contents remains sterile if necessary.

The upper edge of the peripheral bond may comprise the access opening/inlet. The access opening may project out in the plane of the fluid collection bag. The access opening may project away from the fluid collection bag. The access opening may span no more than 30%, 20%, or 10% of the length of the upper edge. The access opening may be positioned at any point on the upper edge, but preferably on one side of the upper edge. The access opening may be any suitable shape, but preferably it is rectangular. Thus, the access opening is spaced from the rest of the bag which makes it easier for the user to locate and also allows it to flex and bend allowing movement of the valve assembly with respect to the fluid collection bag.

The fluid collection bag may comprise a finger hole. The finger hole may be sized to allow a user to put their finger through the finger hole and handle the bag. Thus the finger hole allows the bag to be handled more easily by a user. The finger hole may be provided at an edge of the bag. The finger hole may be provided in the peripheral bond of the bag. The finger hole may be provided at a corner of the bag. The finger hole may be provided at any of the following corners: where the left lateral edge meets the base; where the base meets the right lateral edge; where the right lateral edge meets the upper edge; or where the upper edge meets the left lateral edge. Where the finger hole is provided in the bag, the peripheral bond may be shaped to accommodate the finger hole. The peripheral bond may truncate a corner of the bag to accommodate the finger hole. The peripheral bond may project into the bag to accommodate the finger hole. The finger hole may be any suitable shape but preferably has rounded smooth edges to allow it to be more comfortably grasped by the user. In one example, the finger hole is circular, in another it is stadium shaped, that is a rectangle with semicircles replacing two opposite sides. The finger hole may comprise two elongate straight edges with a length of one quarter to one third, for example 30%, of the height of the fluid collection bag. The elongate straight edges may be aligned parallel to the left and or right lateral edge of the fluid collection bag. The finger hole may have an aspect ratio (length:width) of at least 1:1, 2:1, 3:1, or 4:1, preferably the aspect ratio is 4:1. As an alternative to the finger hole, the fluid collection bag may comprise a series of ridges or otherwise easily gripped surface or feature in the place of the finger hole to allow the bag to be easily handled.

The finger hole may be configured to allow the bag to be rotated. Preferably, one finger hole is positioned at a corner of the bag that is opposite from the access opening/outlet. The finger hole may be adjacent to the base of the fluid collection bag. The finger hole may be closer to one lateral edge (e.g. the right lateral edge) than the access opening/outlet. The access opening/outlet may be closer to the other lateral edge (e.g. the left lateral edge) than the finger hole. Thus, the bag may be hung or held by the finger hole and gravity will drive fluid contained within the bag out through the access opening/outlet.

The fill level indicator may be positioned adjacent to the access opening/inlet/outlet, for example on the same side of the fluid collection bag as the access opening/inlet/outlet and spanning the majority of the height of the fluid collection bag. Thus, the fill level indicator ensures that during filling and draining the fluid within the fluid collection bag is visible as soon as it enters the bag and until it leaves the bag. This helps to ensure the fluid collection bag is completely emptied when draining the bag.

The fluid collection bag may comprise more than one finger hole, for example at least two, three or more finger holes. One, two, three or more, or each finger hole may have any one or more of the features of the finger hole described above. Where there is more than one finger hole, each finger hole may be located at a different corner of the bag. Thus, easy handling of the bag is facilitated through positioning of finger holes or equivalent features which also does not restrict or affect the volume of liquid that can be contained by the bag.

The access opening/outlet may be positioned distal from a finger hole of the bag. Where the access opening/outlet is positioned at or adjacent to an edge of the bag, that edge may be free from finger holes. Where the access opening/outlet is position at or adjacent an edge of the bag, a different edge of the bag (that is the right/left lateral edge) may comprise one or more finger holes. For example, the access opening/outlet may be provided at one corner of the bag and a finger hole may be provided at an opposite corner of the bag. Thus, the finger holes are positioned on edges of the bag that generally do not correspond to the position of the access opening/outlet, this makes handling/emptying of the bag easier as the bag can be rotated and held using the finger holes to allow fluid to leave the bag due to gravity.

The catheter assembly may comprise a fluid reservoir. The catheter assembly may comprise a sleeve configured to enclose the catheter along the length of the catheter from the proximal end to the distal end. The fluid reservoir may be arranged at the proximal end of the catheter. The fluid reservoir may be configured to release wetting fluid into the sleeve to wet the catheter. The sleeve may be in fluid communication with the fluid collection bag.

Accordingly a third aspect of the present invention there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag. Consequently, excess wetting fluid may flow from the sleeve into the fluid collection bag.

Advantageously, the present invention provides for a catheter assembly which benefits from the introduction of wetting fluid at the proximal end of the catheter which reduces the risk of discomfort and injury while using the catheter as the proximal end, which enters the body first, is adequately wetted prior to use. In addition, as the wetting fluid is contained within the sleeve and flows from the proximal end to the distal end, the fluid is used efficiently to wet the whole length of the catheter by prioritising the parts of the catheter that enter the body first. Due to the sleeve being in fluid communication with the fluid collection bag, wetting fluid cannot escape outside the sleeve and as such the user's hands or clothing does not inadvertently contact the wetting fluid which could make handling the catheter difficult. This ensures that the catheter is easy and convenient to use, as well as being discrete and hygienic to dispose of as the wetting fluid is contained within the sleeve/bag.

The sleeve may comprise a flexible plastics material. The sleeve may be liquid impermeable. The sleeve may comprise a thermoplastic polyurethane (TPU) or low-density polyethylene (LDPE). Thus, the sleeve is cheap and easy to produce and can be easily manipulated by the user during use.

The fluid reservoir may be configured to retain a wetting fluid within it. The fluid reservoir may be configured to release wetting fluid to activate the catheter. The wetting fluid may activate the surface of the catheter and render it lubricious. The wetting fluid may be water, such as where the catheter has a hydrophilic surface. Any suitable wetting fluid may be used, for example polar (e.g. water-based) or non-polar (e.g. oil-based) wetting fluids depending on the surface properties of the catheter. The fluid reservoir may be configured to retain at least 5, 10, 15, 25, or 50 ml of wetting fluid.

The fluid reservoir may comprise a deformable, frangible or burstable sachet. A deformable, frangible or burstable reservoir enhances the convenience for a user as fluid can be easily released without additional tools. A sachet in particular can be more easily handled on an assembly line compared to handling fluid itself. The fluid reservoir may comprise a flexible plastics material, for example, a thermoplastic polyurethane (TPU) or low-density polyethylene (LDPE).

The fluid reservoir may be configured to retain at least 5, 10, 15, 25, or 50 ml of wetting fluid. The fluid reservoir may be configured to retain the wetting fluid within it. The fluid reservoir may be configured to retain at least 5, 10, 15, 25, or 50 ml of wetting fluid, most preferably at least 12 ml of fluid. The fluid reservoir may be configured to release wetting fluid into the sleeve; in particular the fluid reservoir may be configured to release wetting fluid between an internal surface of the sleeve and an external surface of the catheter. The fluid reservoir may be configured to retain and/or release excess wetting fluid. Excess wetting fluid is defined herein as more than is normally required to activate/wet the catheter, for example 10%, 20%, 50% or more, in excess of that which would normally be provided to wet the catheter (having regard for example to the length of the catheter and the composition of the wetting fluid). The sleeve may be configured to allow excess wetting fluid to flow into the fluid collection bag. Thus, the reservoir may release an excess of wetting fluid to ensure that the catheter is well wetted and excess fluid can flow into the fluid collection bag ensuring it does not negatively affect subsequent use of the catheter.

The catheter and/or sleeve may be in fluid communication with the fluid collection bag via the valve assembly and preferably connected to the proximal passageway. The proximal passageway may be configured to allow fluid to enter the valve assembly from the distal end of the catheter and optionally via the sleeve. The proximal passageway may be connected to one end of the sleeve which corresponds to the distal end of the catheter. Thus, the proximal passageway allows liquid to pass into the fluid collection bag.

The catheter assembly may be configured to allow fluid to pass directly from the sleeve to the proximal passageway and/or directly from the catheter to the proximal passageway. One lateral side (e.g. a right lateral side) of the catheter may be attached to one side (e.g. a right side) of the proximal passageway. There may be a gap between a different lateral side (e.g. a left lateral side) of the catheter and a respective different side (e.g. a left side) of the proximal passageway. The distal end of the catheter may be within the proximal passageway. The sleeve may be sealed around a periphery of the proximal passageway. As such, fluid travelling within the sleeve is directed into the proximal passageway directly via the gap. In addition, fluid travelling within the catheter passes out the distal end and into the proximal passageway. In other examples, the catheter may be positioned differently and may be attached, for example, to the left side of the proximal passageway.

The catheter assembly may be configured to allow fluid to pass from the catheter into the sleeve. One lateral side (e.g. a right lateral side) of the catheter may be attached to a side (e.g. right side) of the sleeve. A distal end of the catheter may be within the sleeve. A distal end of the catheter may not be within the proximal passageway. The sleeve may be sealed around a periphery of the proximal passageway. As such, fluid travelling within the sleeve is directed into the proximal passageway directly and fluid travelling within the catheter passes out the distal end into the sleeve and then from there into the proximal passageway. In other examples, the catheter may be positioned differently and may be attached to a left side of the sleeve (or indeed a front side or rear side). Furthermore, the distal end of the catheter may be blocked and an outlet may be provided in a lateral side of the catheter to allow fluid to pass out into the sleeve.

The catheter assembly may be configured to allow fluid to pass from the sleeve into the catheter. One lateral side (e.g. a right lateral side) of the catheter may be attached to one side (e.g. a right side) of the proximal passageway. The proximal passageway may be sealed around the catheter on all sides. Consequently, there may be no gap between the proximal passageway and the catheter. As such, fluid may only pass from the sleeve/catheter into the proximal passageway if it is within the catheter. A side, e.g. a lateral side of the catheter may comprise a sleeve intake opening. The sleeve intake opening may be any suitable shape such as oval or circular. The sleeve intake opening may be adjacent to the proximal passageway. The sleeve intake opening may permit the passage of fluid from the sleeve into the catheter. Thus fluid may enter the catheter from the sleeve to join fluid travelling within the catheter. From here, the fluid may pass out the distal end of the catheter and into the proximal passageway. Many other configurations may be possible to achieve the same effect, for example, the sleeve may be attached to the outer sides of the catheter itself.

The sleeve may comprise a reservoir adapter. An end of the sleeve may comprise a reservoir adapter. The reservoir adapter may be configured to engage the fluid reservoir. The reservoir adapter may be configured to receive wetting fluid from the fluid reservoir. The reservoir adapter may be configured to detach from the fluid reservoir. The reservoir adapter may be configured to permit the catheter to pass through the reservoir adapter.

According to a broad aspect of the present invention, there is provided catheter assembly comprising a fluid reservoir, a catheter which comprises a proximal end for insertion into the body and a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

The catheter assembly may be a packaged catheter assembly comprising a pouch containing the fluid reservoir and catheter. According to a fourth aspect of the present invention, there is provided a packaged catheter assembly comprising a fluid reservoir, a catheter, and a pouch containing the fluid reservoir and catheter, wherein the catheter comprises a proximal end for insertion into the body, a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

Thus, wetting fluid is efficiently and easily introduced to wet the catheter as the reservoir adapter retains the fluid reservoir at one end of the sleeve. This ensures that wetting fluid is not wasted and also that wetting fluid is less likely to leak out and wet the external surface of the sleeve or pouch which may cause discomfort for the user.

The sleeve may comprise a proximal end and a distal end corresponding to the proximal end and distal end of the catheter respectively. The proximal end of the sleeve may comprise the reservoir adapter. The reservoir adapter may be positioned at or adjacent to the proximal end of the catheter. Thus, the reservoir is adapted to deliver wetting fluid to the proximal end of the catheter first, this ensures that the part of the catheter first introduced into the body is most likely to be adequately wetted. This reduces the risk of injury or discomfort during use of the catheter.

The reservoir adapter may annular, for example it may be an O-ring. The reservoir adapter may comprise a bore therethrough. The bore may be sized to allow the catheter to pass through the reservoir adapter. The bore may be sized to allow the catheter and wetting fluid to pass through the reservoir adapter. The reservoir adapter may be configured to inhibit passage of the catheter through the reservoir adapter/bore while the fluid reservoir is engaged by the reservoir adapter. The fluid reservoir may be configured to block the catheter from passing through the reservoir adapter/bore. The fluid reservoir may be configured to block the catheter from passing through the reservoir adapter/bore if the fluid reservoir is engaged by the reservoir adapter. Thus, the catheter may be withdrawn from the sleeve through the reservoir adapter and bore, but only once the fluid reservoir is removed from the reservoir adapter. This helps to protect the catheter from dirt and damage prior to use.

The reservoir adapter may be configured to engage the fluid reservoir by any suitable means, such as push-fit, bayonet fit, or screw-fit engagement. The reservoir adapter may have an internal size and shape that matches the fluid reservoir. Thus, the fluid reservoir may be easily push-fit into the reservoir adapter.

The fluid reservoir may comprise a housing. The housing may be configured to surround the sachet, preferably the housing is tight fitting on all sides of the sachet. The housing may be configured to direct wetting fluid released from the sachet to wet the catheter. The housing may be fluid impermeable. The housing may be flexible. The housing may be formed from any suitable material such as silicone or a flexible plastics material like thermoplastic polyurethane (TPU) or low-density polyethylene (LDPE). The housing may be substantially the same shape as the sachet, for example rectangular. The housing may have rounded edges, this can help to ensure fluid does not get trapped in corners of the housing.

The reservoir adapter may engage the housing. The reservoir adapter may engage the housing via a wetting tube positioned at the proximal end of the sleeve. The housing may be integrally formed with the wetting tube. The housing may be a separate object from the wetting tube. The housing may be engaged by the wetting tube in a variety of ways such as push-fit, screw-fit or through an adhesive or weld, preferably the housing is push-fit into the wetting tube. The sleeve may extend around the wetting tube. The reservoir adapter may be fitted around the sleeve. The reservoir adapter may be fitted around the wetting tube. The reservoir adapter may be fitted around the housing. The reservoir adapter may urge any two or more of the sleeve, wetting tube and housing into engagement with one another. The Thus, the reservoir adapter can provide a simple but effective fluid tight seal between the fluid reservoir and sleeve.

The reservoir adapter may have an inner diameter that is less than an outer diameter of the wetting tube. The reservoir adapter may have an inner diameter that is less than an outer diameter of the housing. The reservoir adapter may be formed of a material that is more flexible than the wetting tube. The sleeve may be formed of a material that is more flexible than the reservoir adapter and/or wetting tube. Thus, the reservoir adapter stretches to accommodate the wetting tube and does not significantly reduce the size of the wetting tube when it is engaged by the reservoir adapter. This ensures fluid flow from the fluid reservoir to the sleeve is not unnecessarily inhibited.

The fluid reservoir may be configured to release wetting fluid when activated by a user. The fluid reservoir may comprise an activation marker. The fluid reservoir may be configured to release wetting fluid upon activation of the activation marker. The activation marker may be on a surface of the fluid reservoir, such as on the housing. The activation marker may comprise a region of the fluid reservoir that is identifiable to the user, for example through visual and/or tactile feedback. Preferably, the activation marker may be at least visually recognisable, for example a different colour, shade of colour, or pattern, to the rest of the fluid reservoir. Thus, the activation marker is easily recognisable while the fluid reservoir is engaged by the reservoir adapter.

The pouch may comprise two walls (a front wall and a rear wall). The pouch may comprise a peripheral seal between the two walls. When the pouch is sealed it may be in a closed configuration. The peripheral seal may comprise any one or more of: a weld; mechanical seal; heat seal; pressure seal; adhesive; solvent bond; ultraviolet bond; ultrasonic weld; laser weld; impulse weld; or friction weld. The peripheral seal may be re-sealable, for example through a resealable adhesive, an additional adhesive that is activated prior or during resealing, or a hook and loop/hook and hook fastener. As outlined above, the peripheral seal may provide a sterile seal. Both the fluid collection bag and peripheral seal of the pouch may provide a sterile seal. Thus the peripheral seal can be easily constructed to suit the specific needs of the pouch and ensure the contents remains sterile within the pouch. Advantageously, additional protection is provided to ensure the catheter remains sterile allowing the pouch to be opened to provide a sterile surface which, if used and made dirty, does not affect the sterility of the catheter.

The peripheral seal may form one or more edges of the pouch. A folded edge may comprise the fold between the two walls (the first wall and the second wall). All edges other than the folded edge may be formed by the peripheral seal. Preferably, the peripheral seal comprises all edges of the pouch. The peripheral seal may comprise a base, a left lateral edge, right lateral edge and an upper edge. The left and right lateral edges may be defined as the left and right edges when viewing the pouch in the closed configuration with the front wall in front of the rear wall, the base at the bottom and the upper edge at the top.

The pouch may have a width defined as the distance between the left lateral edge and right lateral edge of the pouch of at least 60, 70, 80, 90, 100, 120, 140, 160 or 180 mm. The pouch may have a width of no more than 200, 180, 160, 140, 120, 100, 90, 80, or 70 mm. Preferably, the width is between 60-110 mm, and most preferably about 90-110 mm, for example 105 mm. The pouch may have a height defined as the distance between the upper edge and base (i.e. the height when in the closed configuration) of at least 60, 70, 80, 90, 100, 120, 140, 160, 180, 200, 220, 240 or 250 mm. The pouch may have a height of no more than 250, 240, 220, 200, 180, 160, 140, 120, 110, 100, 90, 80 or 70 mm. Preferably, the height is between 100-250 mm, or 120-170 mm and most preferably it is about 1500 mm. Thus, the pouch is a compact shape when closed so that it is convenient and discreet for the user to carry, for example it is easier to fit in a bag or pocket.

The folded edge may be configured to resist being broken. A frangible portion of the peripheral seal may form one or more edges of the pouch that do not comprise the folded edge, in particular, all the edges other than the folded edge may form the frangible portion. The frangible portion may be configured to break before the folded edge. The pouch may comprise only one folded edge.

The pouch may contain any one or more of the fluid collection bag and catheter. The fluid collection bag may be provided in a stowed configuration while inside the pouch. Thus, the size of the fluid collection bag is minimised inside the pouch and this assists the user in removing the bag and other elements of the packaged catheter assembly from the pouch.

The pouch may comprise a flexible plastics material. For example, polypropylene (PP), polyethylene terephthalate (PET), low density polyethylene (LDPE), metalized polyester (MET PET) or orientated polypropylene (OPP). The pouch may be formed from an opaque material. The pouch may have a printed appearance or may be matte. This reduces the impression that it is a medical device and thus make it more discreet to carry.

The pouch may comprise an interaction region defining a first point of contact for a user of the packaged catheter assembly. The interaction region may facilitate user access to the contents of the pouch. Actuation of the interaction region may create an opening in the pouch. The interaction region may be a break-away region such as a peel-away or tear-away region configured to be at least partially separated from the pouch. The catheter may be withdrawn through the opening. The catheter may be withdrawn through the opening. Before withdrawal of the catheter from the pouch, the opening may comprise a sterile seal. The sterile seal may be broken by actuation of the interaction region, for example by peeling a peel-away region from the pouch.

The break-away region may form a part of the walls of the pouch, for example, the break-away region may form a part of the front wall and/or rear wall. Preferably, the break-away region is positioned adjacent to a lateral edge (e.g. right lateral edge) of the pouch. The break-away region may be any suitable shape or size, for example, rectangular, circular, ellipsoidal or irregularly shaped. Preferably, the break-away region is rectangular. The break away region may be defined by a tear line along which the pouch preferentially tears. The tear line may comprise a line of weakness in the walls of the pouch. The tear line may be formed by any suitable means, for example laser scoring. The tear line may span between two edges of the pouch, for example the upper edge and base. The tear line may be present on the front and/or rear wall of the pouch, preferably just the front wall. A tear start may be provided at a first end of the tear line. The tear start may comprise a notch, or area of weakness, in one or both of the walls of the pouch. The tear start may define the first part of the pouch to be torn. A tear stop may be provided at a second end of the tear line. The tear stop may act to prevent or reduce the likelihood of the tear-away region being completely separated from the remainder of the packaging. Thus, the break-away region can be used to form an opening in the pouch in a controlled manner. In addition, the tear line can only span one wall of the pouch which prevents the break-away region from becoming fully separated from the pouch which may be a nuisance for the user as they would then need to retain and dispose of an additional loose item.

The tear line may be flexibly positioned on the pouch. The tear line may span between two points on the edge of the pouch. The tear start may be positioned on an edge of the pouch. The tear stop may be positioned on an edge of the pouch. The tear start and tear stop may be positioned on opposite edges of the pouch, preferably the upper edge and base respectively. The tear line may be positioned off-centre with respect to the width of the pouch. Preferably, the tear line is positioned 70-90% across the width of the pouch from one lateral edge to the other, (e.g. left lateral edge to right lateral edge). The break-away region may therefore correspond to 30-10% of the width of the pouch. As such, a break-away region is formed which is a minority of the width of the pouch and this ensures that the contents of the pouch do not fall out in an uncontrolled manner as soon as the pouch is opened.

The break-away region may be the same colour, a different shade of colour or a different colour to the rest of the pouch. Thus, the break-away region can either be made more discreet or more obvious depending on the needs of the packaged catheter assembly.

The break-away region may comprise a pull-ring. The pull ring may be arranged adjacent to the tear start. The pull ring may be sized to allow a finger to pass through it. The pull ring may be configured to tear the pouch along the tear line. Thus, the pull ring makes it easier for the user to open the pouch by providing a ring which they can easily grip and pull, of course a ring itself may not be necessary and instead any feature the user can easily grasp could be used.

The pull ring may be a different colour, or shade of colour, from the walls of the pouch. Thus, it is more easily identifiable for the use.

The break-away region may comprise an opening marker configured to provide guidance to the user about how to open the pouch. The opening marker may direct the user to tear the pouch along the tear line, for example by grasping and pulling on the pull ring. The opening marker may comprise an arrow. The arrow may point from the tear start towards the tear stop along the tear line. The opening marker may provide visual guidance to the user, or may provide tactile guidance or otherwise for example using a region of the pouch that is a different texture to the rest of the pouch.

The fluid collection bag may be contained within the pouch. The fluid collection bag may be provided in a stowed configuration, preferably while inside the pouch. The fluid collection bag may be configured to remain in the stowed configuration. In the stowed configuration, the fluid collection bag may be folded or rolled to reduce its size in any one dimension. The stowed configuration may be a folded or rolled configuration. Preferably, the fluid reservoir is folded along its width and height when in the stowed configuration. Thus, the fluid collection bag is stored in a compact manner that allows the pouch to also be a small size and convenient for the user to carry on their person.

The access opening may be provided in a folded configuration within the pouch. The valve assembly may overlap the fluid collection bag within the pouch. The fluid reservoir may be contained within the pouch. The fluid reservoir may be positioned adjacent to the valve assembly in the pouch, for example beneath the valve assembly. The catheter may be provided in a curved and/or coiled configuration. The distal end of the catheter may be arranged adjacent to the valve assembly/proximal passageway. The proximal end of the catheter may be arranged adjacent to the fluid reservoir/reservoir adapter/wetting tube. The sleeve may comprise a turn between the wetting tube and the proximal end of the catheter. The turn may be at least 90 degrees, 120 degrees, 150 degrees or 180 degrees, preferably the turn is 180 degrees. Preferably, the turn is a hairpin turn. Thus, the packaged catheter assembly is arranged in a compact manner that is easy to access and use.

The fluid reservoir may be in a position within the pouch corresponding to the tear line. The fluid collection bag may be in a position within the pouch corresponding to the tear line. Thus, when the tear line is torn, the fluid reservoir and fluid collection bag are accessible for the user to grasp and remove from the pouch.

The fluid collection bag may comprise a retaining element. The retaining element may be configured to receive at least part of the catheter to stow the catheter after use.

According to a fifth aspect of the invention, there is provided a catheter assembly comprising a fluid collection bag and a catheter, the fluid collection bag comprising a retaining element configured to receive at least part of the catheter to stow the catheter after use.

Advantageously, the retaining element ensures the catheter is stowed which makes the catheter assembly more hygienic to store and dispose of after use without having to carry a separate item, such as a bag, to stow/store the catheter in.

The catheter may be configured to be coiled when stowed. Thus, the catheter's size is minimised to ensure it is stowed efficiently.

The retaining element may be permanently attached to the fluid collection bag. Thus, the retaining element is unlikely to be lost or become separated from the fluid collection bag, this makes it more convenient and easier to use.

The retaining element may comprise a pocket on the outside of the fluid collection bag. The pocket may be configured to receive a majority of the length of the catheter after use. The pocket may comprise a flexible sheet material, for example any one of the materials that were described in relation to the fluid collection bag above. Preferably, the pocket comprises a liquid impermeable material. Thus, the pocket prevents liquid passing from the used catheter to the outside of the pocket where it could cause a hygiene risk if it comes into contact with the user or another object.

The pocket may have any suitable shape and size to stow the catheter, for example, the pocket may span the entire width (or length) of the fluid collection bag. In other embodiments, the pocket may not span the entire width (or length) of the fluid collection bag. Preferably, the pocket spans the entire width of the fluid collection bag. Preferably, the pocket spans a majority of the height of the fluid collection bag. The pocket may span 50-80% of the height of the fluid collection bag, for example at least 50%, 60%, or 70% of the height of the fluid collection bag and most preferably 60%. Thus, the pocket can be used to safely store the catheter and prevent inadvertent contact with it by the use.

The pocket may be attached to the fluid collection bag by the peripheral bond. The pocket may be joined to a base of the fluid collection bag. The pocket may be joined to left and/or right lateral edges of the fluid collection bag. Thus, the pocket can be easily attached to the bag in a single step when forming the peripheral bond.

The retaining element may comprise a strap. The strap may be configured to seal the catheter against the fluid collection bag after use. The strap may comprise a strip of material. The strap may be configured to seal to the fluid collection bag. The strap may be configured to seal to the catheter. The strap may be configured to seal to itself. The strap may seal to the fluid collection bag/catheter/itself via any suitable adhesive, for example an epoxy resin or alternatively a silicone-based adhesive. The strap may be re-sealable, for example through a resealable adhesive, an additional adhesive that is activated prior or during resealing, or a hook and loop/hook and hook fastener. Thus, the strap can be configured to seal to a variety of different surfaces to ensure the catheter can be easily stowed.

A part of the strap may be permanently attached to the fluid collection bag. For example, a part of the strap may be permanently attached to the fluid collection bag by any one or more of: a weld; mechanical seal; heat seal; pressure seal; adhesive; solvent bond; ultraviolet bond; ultrasonic weld; laser weld; impulse weld; or friction weld. Thus, as part of the strap is attached to the bag it is easier to retain the catheter against the bag. Preferably, one end of the strap (a first end) is attached to the bag. Thus, the rest of the strap can be used most efficiently to stow the catheter.

A part of the strap may be configured to temporarily attach to the fluid collection bag. A part of the strap may be re-sealable. Preferably, one end of the strap is re-sealable. A second end of the strap may be the re-sealable end. Thus, the catheter can be arranged in the stowed configuration and then the strap used to retain it in place by attaching the second end of the strap to the bag. This makes the strap more convenient to use as, for example, the user doesn't need to feed the catheter through a gap between the strap and bag.

The retaining element may be configured to restrict the size of the fluid collection bag. Thus, the fluid collection bag can be more convenient and discreet for the user to transport, store, use and dispose of. The fluid collection bag may comprise one or more stowed configurations, for example a rolled configuration and/or a folded configuration. The strap may be configured to retain the fluid collection bag in a stowed configuration. A part of the strap (the first end) may be configured to seal to one panel of the fluid collection bag (e.g. the front panel). A different part of the strap (the second end) may be configured to seal to another panel of the fluid collection (e.g. the rear panel). Thus, the strap may retain the fluid collection bag in a folded configuration, which can help control and reduce leaks of liquid out of the bag.

In the folded configuration, the fluid collection bag may comprise a fold (in an upper region of the fluid collection bag, for example adjacent to the upper edge and/or neck). Thus, the passage of liquid towards the inlet from inside the bag is reduced by the fold helping to prevent leaks. The strap may be configured to extend around the fold. Thus, the strap ensures the fold is maintained.

The fluid collection bag may comprise a rolled configuration. The fluid collection bag may be arranged in the rolled configuration prior to use. For example, the fluid collection bag may be provided in the rolled configuration in a sterile sealed pouch. The strap may extend (all the way) around the fluid collection bag in the rolled configuration. The strap may seal to itself when retaining the fluid collection bag in the rolled configuration. Preferably, one end (e.g. the second end) may be configured to seal to another part of the strap. Thus, the fluid collection bag's size is minimised making it more convenient and discreet to store prior to use.

As mentioned above, the catheter assembly may comprise a pouch and may a packaged catheter assembly. The retaining element may be configured to restrict movement of the fluid collection bag within the pouch, for example, the retaining element may retain the fluid collection bag in a particular part of the pouch. Thus, the fluid collection bag can be more easily accessible to the user when opening the pouch. The retaining element may be configured to (temporarily) attach the fluid collection bag to a part of the pouch, for example a wall of the pouch. The retaining element may be configured to retain the fluid collection bag adjacent to an opening formed in the pouch. Thus, the fluid collection bag is attached to the pouch wall next to the opening which makes it easier to identify and access.

The retaining element may comprise a release mechanism. The release mechanism may be configured to release the retaining element such that the fluid collection bag can be released from the pouch and/or unrolled/unfolded prior to use. The release mechanism may comprise a gripping hole or other tactile element to make it easier to use. Thus, the retaining element can be easily deactivated and the fluid collection bag used.

The release mechanism may comprise a pull-tab. The pull-tab may comprise a tab configured to be pulled by the user to release the retaining element. One end (the second end) of the strap may be the pull-tab. Thus, a tab is easy for the user to grasp and intuitive to use.

The release mechanism may comprise a release marker. The release marker may comprise a logo, marking, a different colour/shade of colour, or different texture/tactile feel than the rest of the fluid collection bag/release mechanism/retaining element. The release marker may be recognisable visually. The release marker may be recognisable through touch. The release marker may be configured to instruct the user how to activate the release mechanism. The release maker may comprise a pull-marker. The pull-marker may be configured to instruct the user to pull the release mechanism/pull-tab in a particular direction, for example, the pull-marker may comprise a directional arrow or point in that direction.

The pouch may comprise one or more side walls, for example two side walls. The side walls may be configured to allow the front and rear walls to separate. Each side wall may comprise a lateral side of the pouch. The side walls may be elongate in a direction parallel to the length of the pouch. The side walls may be triangular, i.e. substantially triangular. The side walls may extend between the front and rear walls of the pouch. The side walls may extend along the left and/or right lateral edge of the pouch. The side walls may not extend along one or more edges of the pouch, for example the base and/or upper edge of the pouch. Thus, the side walls can be used to increase the internal volume of the pouch while minimising the size of the front and rear walls.

The folded edge may be configured to resist being broken. A frangible portion of the peripheral seal may form one or more edges of the pouch that do not comprise the folded edge, in particular, all the edges other than the folded edge may form the frangible portion. The frangible portion may be configured to break before the folded edge. The pouch may comprise only one folded edge.

The pouch may contain any one or more of the fluid collection bag and catheter. The fluid collection bag may be provided in a stowed configuration while inside the pouch. Thus, the size of the fluid collection bag is minimised inside the pouch and this assists the user in removing the bag and other elements of the packaged catheter assembly from the pouch.

The pouch may comprise a flexible plastics material. For example, polypropylene (PP), polyethylene terephthalate (PET), low density polyethylene (LDPE), metalized polyester (MET PET) or orientated polypropylene (OPP).

The pouch may comprise an interaction region defining a first point of contact for a user of the packaged catheter assembly. The interaction region may facilitate user access to the contents of the pouch. Actuation of the interaction region may create an opening in the pouch. The interaction region may be a break-away region such as a peel-away or tear-away region configured to be at least partially separated from the pouch. The catheter may be withdrawn through the opening. The catheter may be withdrawn through the opening. Before withdrawal of the catheter from the pouch, the opening may comprise a sterile seal. The sterile seal may be broken by actuation of the interaction region, for example by peeling a peel-away region from the pouch.

The break-away region may form a part of the walls of the pouch, for example, the break-away region may form a part of the front wall. Preferably, the break-away region forms a majority of the front wall. The break-away region may be any suitable shape or size, for example, rectangular, circular, ellipsoidal or irregularly shaped. Preferably, the break-away region is the same shape as the pouch, for example rectangular. The break-away region may comprise a base, left lateral edge, right lateral edge and upper edge that form the edges of the interaction region. The edges of the interaction region may correspond to the respective edges of the pouch. The break-away region may comprise a width between its left lateral edge and right lateral edge. The width of the break-away region may be at least 50%, 60%, 70%, 80% or 90% of the width of the pouch, for example, between 50-100%, 70-90% or 80-90% of the width of the pouch, most preferably the break-away region has a width of 85% of the width of the pouch. The break-away region may comprise a height between its base and upper edge. The height of the break-away region may be at least 50%, 60%, 70%, 80% or 90% the height of the pouch, for example between 50-100%, 60-90% or 70-80% of the height of the pouch, most preferably 75% of the height of the pouch. The break-away region may be positioned centrally with respect to the width and/or height of the pouch. Alternatively, the break-away region may have a different shape to the pouch and may not be positioned centrally with respect to the width and/or height of the pouch. Thus, the break-away region can be configured in many different ways to provide easy access to the contents of the pouch.

The break-away region may comprise rounded edges. Thus, the break-away region has a smoother and less jagged appearance.

The break-away region may formed of the same material as the walls of the pouch. This may ensure that the pouch has a consistent feel to the user and also that one area of the pouch is not weaker than the rest. Of course, in some embodiments, the break-away region may be formed of a different material to the rest of the pouch, for example where certain mechanical properties/tactile feel of the break-away region are desired.

The break-away region may be the same colour, a different shade of colour or a different colour to the rest of the pouch. Preferably, the break-away region is a different shade of colour, or different colour, to the rest of the pouch. This makes it more visually recognisable.

The pouch may comprise an access aperture. The access aperture may be positioned in one wall of the pouch, for example, the front wall. The break-away region may be configured to cover and/or seal the access aperture. The break-away region may be attached to the pouch by any suitable bonding/sealing method, for example chemical adhesives, chemical bonding, ultrasonic bonding, or mechanical bonding. This arrangement is preferred where the break-away region is a peel-away region, as the sealing/bonding method can be selected to allow the peel-away region to be peeled away. The break-away region may be formed from the same piece of material as a wall of the pouch. A line of weakness or tear-line in the pouch may define the break-away region. This arrangement is preferred where the break-away region is a tear-away region, as the tear-away region can be torn away along the line of weakness/tear line. Thus, the user to form an opening in the pouch/reveal the access aperture by separating the break-away region from the pouch.

An edge of the break-away region may be both part of the break-away region and part of a wall (e.g. front wall) of the pouch. Thus, a fold-line may be defined at the edge of the break-away region. The break-away region may pivot to allow access to the pouch. The break-away region may be configured to remain connected to the pouch. This reduces the number of items that the user must worry about during use of the catheter assembly which makes it more convenient and easier to use.

The fold-line may correspond to the upper edge of the break-away region, thus the break-away region swings upwards away from the base of the pouch. The fold-line may correspond to the base of the break-away region, thus the break-away region swings down away from the upper edge of the pouch. In some embodiments, the fold-line may correspond to a lateral edge of the break-away region allowing the break-away region to provide a door into the pouch. Thus, the break-away region can be flexibly configured to provide access to the pouch in many different ways.

The fluid collection bag may be attached to the break-away region, for example by the retaining element as described above. The break-away region may be configured to remove the fluid collection bag from the pouch during opening of the pouch. This makes it easier to access the fluid collection bag.

The pouch may comprise a tab. The tab may extend from an edge of the pouch/break-away region. The tab may be configured to create an opening in the peripheral seal of the pouch, for example through (partial) separation of the break-away region and pouch. Thus, the pouch can be more easily opened.

The tab may extend from one (i.e. the front or rear) wall of the pouch. The tab may be configured to fold down to overlie a (rear or preferably front) wall of the pouch. The tab may be configured to seal to a (rear or preferably front) wall of the pouch. Thus, the folded tab does not extend out and increase the bulk of the pouch.

The tab may span the majority of the width of the pouch, for example the entire width of the pouch. The tab may be triangular. A tip of the tab may extend down about half the height of the pouch. The tab may give the closed pouch the appearance of a closed envelope. Thus, the pouch has a familiar appearance that the user will recognise and be able to open easily.

Preferably, one wall comprises the break-away region. A tab may be provided on the break-away region. A tab may extend from an edge of the pouch. The tab may be configured to be separate from the pouch to create an opening in the peripheral seal of the pouch. Advantageously, the tab extends from the pouch and so is easy to grasp by the user. This makes opening the pouch easy even for users with reduced mobility.

The tab may comprise a front opening tab. The front opening tab may be attached to the break-away region. The front opening tab may overlie the pouch and preferably the front opening tab overlies an edge of the pouch. The front opening tab may be smaller an edge of the pouch. Thus, the tab can be easily identified and separated to open the pouch.

The tab may comprise an opening marker. The opening marker may be configured to provide a visual/tactile guide to the user to prompt them to open the pouch. The opening marker may comprise an arrow, or a pointed shape such as a triangle. The opening marker may prompt the user to pull or move the tab to open the pouch. The arrow may point in the direction the tab should be opened. Preferably, an opening marker is provided on the tab connected to the break-away region. Thus, the pouch is more user friendly.

The tab may extend from an edge of the pouch that corresponds to a frangible portion of the peripheral seal. Thus, the tab is suitably located to ensure the frangible portion breaks when the tab and pouch are separated.

The tab may be configured to provide a handling point for the pouch. Thus, the tab can be used to conveniently handle and move the pouch as may be required.

The tab may be configured to allow to the pouch to be closed. This feature may preferably be used in combination with a peripheral seal that is re-sealable. Thus, the tab provides a handling point for the user to close the pouch, this can be particularly useful after using the catheter as the rest of the pouch may be dirty or soiled and so is undesirable to touch.

The tab may be any suitable size or shape, such as rectangular, square, circular, elliptical, or irregular in shape. Various shapes and sizes of a tab are discussed below but of course each tab may be the same or different shapes.

The tab may extend from the break-away region, preferably the tab may extend from the centre of an edge of the break-away region. The tab may extend from the base or upper edge of the break-away region. The edge of the break-away region may be slanted towards the tab. The tab may span at least 30%, 40%, 50%, 60%, or 70% of the width of the break-away region. The tab may span no more than 80%, 70%, 60%, 50% or 40% of the width of the break-away region. The tab may have a length that is similar to its width. The tab may have any suitable shape, such as rectangular, triangular or semi-circular. Thus, the tab is easily identifiable at the end of the break-away region.

A tab may be curved. Preferably the tab is curved in a direction away from the plane of the pouch. Thus, the tab may naturally bend away from the pouch which makes it easier to grasp and separate the tab from the pouch. A wall of the pouch may curve away from the tab. Thus, the pouch bends away from the tab which also makes it easier to grasp and separate the tab from the pouch.

The tab may comprise a gripping element, for example a gripping hole. The gripping hole may provide a grippy surface or feature. The gripping hole may comprise an aperture through the tab. The gripping hole may be any suitable size or shape, for example, rectangular, square, circular, elliptical. In one embodiment, the gripping hole is in the shape of a stadium, that is a rectangle with semicircles replacing two opposite sides. The parallel sides of the gripping hole may be parallel to the base and top of the tab. Thus, the addition of a gripping hole to a tab allows the user to easily grasp it, of course a hole itself may not be necessary and instead any feature that allows the user to easily grasp the tab could be used.

An edge (e.g. base or upper edge) of the pouch may comprise a gripping hole, for example a gripping hole. The gripping hole of the pouch may have any one or more of the features of the gripping hold of the tab described above. Preferably, the gripping hole of the pouch has the same size/shape as the gripping hole of the tab. The gripping hole of the tab may be in overlap with the gripping hole of the pouch. Thus, the user can more easily separate the pouch and the tab.

The pouch may be divided into first and second compartments. The fluid reservoir and at least a portion of the catheter may be arranged in the first compartment. The fluid collection bag may be connected to the catheter and arranged in the second compartment. The fluid reservoir may be configured to release the wetting fluid into the first compartment to wet the catheter. The pouch may comprise an interaction region operable by a user to form an opening in the pouch allowing access to at least the second compartment.

According to a sixth aspect of the invention, there is provided a packaged catheter assembly comprising a catheter, a fluid reservoir comprising wetting fluid, and a pouch, wherein: the pouch is divided into first and second compartments; the fluid reservoir and at least a portion of the catheter are arranged in the first compartment; and a fluid collection bag is connected to the catheter and is arranged in the second compartment; and the fluid reservoir is configured to release the wetting fluid into the first compartment to wet the catheter; and the pouch comprises an interaction region operable by a user to form an opening in the pouch allowing access to at least the second compartment.

With this arrangement, a substantial amount, if not all, of the wetting fluid remains in the first compartment upon release of the wetting fluid from the fluid reservoir. Advantageously, this means that the fluid collection bag remains dry and free from wetting fluid prior to use. Beneficially, for the user, in particular for users with limited dexterity, this means handling and re-packaging of the bag during and after use is considerably easier than if the bag is wetted with wetting fluid.

Further advantageously, the wetting fluid is retained in the first compartment comprising at least a portion of the catheter and is not distributed amongst the components in the second compartment of the pouch. This may be the case even where the pouch is vigorously shaken to distribute the wetting fluid within it. This means that all of the wetting fluid, or at least a substantial amount of the wetting fluid, is available to wet the catheter prior to use, as is intended. Beneficially, this arrangement ensures that there is sufficient wetting fluid to activate the whole catheter surface, therefore, preventing discomfort and injury during use.

The opening may be provided in the pouch by separating the break-away region from the pouch to reveal the access aperture as described above.

The opening may allow access to the first compartment. Access to the first compartment may be via the second compartment. Beneficially, this means that the fluid collection bag is withdrawn from the pouch through the opening before the catheter. Thus, the user does not need to touch the wetted catheter before touching the fluid collection bag, therefore, preventing accidental wetting of the fluid collection bag during withdrawal of the components from the pouch.

The pouch may comprise an internal wall. The internal wall may define the first and second compartments. The first compartment may comprise the rear wall and internal wall. The second compartment may comprise the front wall and internal wall. The internal wall may be the same material as the front wall and or rear wall of the pouch. The internal wall may be fluid impermeable. Thus, the compartments can be simply defined by including an additional wall within the pouch and further the wall prevents or at least inhibits liquid from passing between the compartments.

The internal wall may be arranged to inhibit wetting fluid from the fluid reservoir wetting the second compartment and the fluid collection bag arranged therein. Advantageously, this means that the fluid collection bag remains dry prior to use. Beneficially, for the user, in particular for users with limited dexterity, this means handling and re-packaging of the bag during and after use is considerably easier than if the bag is wetted with wetting fluid.

Moreover, this arrangement allows a user to vigorously shake the pouch after wetting fluid is released from the fluid reservoir, therefore, ensuring distribution of wetting fluid to the catheter without the risk of wetting the fluid collection bag. Beneficially, this ensures that there is sufficient wetting fluid to activate the whole catheter surface, therefore, preventing, or at least significantly reducing, discomfort and injury during use.

The internal wall may be attached to the pouch by the peripheral seal. The internal wall may span the entire width of the pouch. The internal wall may span a majority of the height of the pouch, for example at least 50%, 60%, 70%, 80%, or 90% of the height of the pouch, most preferably the internal wall spans 80% of the height of the pouch. The internal wall may extend from the base of the pouch. Each compartment may be planar. The first and second compartment may be arranged parallel to one another. Thus, the internal wall ensures that both the first and second compartments are adequately sized and further the pouch may be sealed with the internal wall within by forming the peripheral seal in a single step.

The internal wall may comprise a separating edge. The separating edge may not form part of the peripheral seal. Access between the first and second compartments may be provided at or adjacent to the separating edge. The separating edge may be a free edge in that it is not sealed to either the front or rear walls of the pouch. In such embodiments, access may be provided between the first and second compartments at all points along the separating edge.

The separating edge may comprise a cut-away region. The cut-away region may be cut into the internal wall/separating edge. The cut-away region may be centrally located across the width of the pouch. The cut-away region may have a size which is substantially equal to, or greater than, the diameter of the catheter. The catheter may be arranged such that before withdrawal of the catheter from the pouch, a portion of the catheter (preferably a portion at or adjacent the distal end) is positioned within the cut-away region. Preferably, the cut-away region may have a size which is substantially equal to, or greater than, the diameter of the catheter tube, and that before withdrawal of the catheter from the pouch, the catheter tube is positioned within the cut-away region. The cut-away region may span at least 10%, 20%, 30%, 40%, 50%, or 60% of the width of the pouch and/or break-away region. The cut-away region may have any suitable shape or size, for example, semi-circular, triangular, rectangular, or irregularly shaped. In one example, the cut away region is trapezoid. The cut-away region may be an inverted (isosceles) trapezoid. The cut-away region may have an aspect ratio (length:width) of 1:2.

Advantageously, this arrangement provides for a connection between the fluid collection bag and the catheter which does not impact the ability of the pouch to retain the wetting fluid in the first compartment. Thus, the fluid collection bag remains dry prior to use. Moreover, typically, a catheter is of sufficient length such that the distal end of the catheter is not required to be inserted into the body during use. Thus, in embodiments where a portion of the distal end of the catheter tube is positioned in the cut-away region, it is not essential for this portion of the catheter to be wetted by the wetting fluid. As such, the catheter tube can be positioned within the cut-away region without having a detrimental effect on the use of the catheter.

The separating edge may be sealed along part of its length to the front or rear wall (preferably rear wall). Thus, access is only provided along a part of the separating edge. Access between the first and second compartments may be provided (solely) by the cut-away region. This can help to control and restrict access between the compartments.

The opening/access aperture may be provided in a wall of the pouch that defines the second compartment, e.g. the front wall. The break-away region may form part of the second compartment. Thus, when the break-away region is separated from the pouch, access to the second compartment is provided.

The catheter may be arranged in the first compartment in any suitable configuration. The catheter may be arranged in a curved/coiled configuration. Thus, the catheter is less likely to suffer damage from kinking etc.

The catheter may comprise a sleeve configured to enclose part of the catheter. Preferably, the sleeve is configured to enclose only part of the length of the catheter. The sleeve may be tubular. The sleeve may comprise a flexible plastics material. The sleeve may comprise perforations about its circumference. The sleeve may comprise a line of weakness (provided by the perforations). The sleeve may be configured to separate into two tubular handling elements. Thus, the sleeve is cheap and easy to produce and can be easily manipulated by the user during use.

The tubular sleeve may be provided in the cut-away region of the internal wall. Thus, the sleeve provides a plug which prevents liquid from inadvertently passing along the outside of the catheter and through the cut-away region into the second compartment during wetting.

The catheter assemblies/packaged catheter assemblies of the first to sixth aspects may include any one or more features of a catheter assembly/packaged catheter assembly as defined in general/broad terms, or according to any other of the first to fourth aspects set out above. The catheter assemblies/packaged catheter assemblies of the first to sixth aspects may comprise any of the optional features of the others of the first to sixth aspects without necessarily including all the features required of them. That is to say, an optional feature which happens to be set out following one particular aspect does not necessarily apply only to that aspect, so, for example, the disclosure provides for a packaged catheter assembly comprising a pouch containing the catheter assembly of the first aspect, that is, a catheter assembly comprising a catheter having a proximal end for insertion into the body and a distal end, a valve assembly, and a fluid collection bag arranged to receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag; wherein the fluid collection bag comprises two side panels as described in relation to the second aspect and wherein the pouch comprises a break-away region comprising a pull ring as described in relation to the third aspect.

According to a seventh aspect of the present invention there is provided a method of manufacturing a catheter assembly comprising the steps of providing a catheter having a proximal end for insertion into the body and a distal end and a fluid collection bag, providing a valve assembly and arranging it between the catheter and fluid collection bag such that the fluid collection bag may receive fluid from the distal end of the catheter via the valve assembly, wherein the valve assembly comprises: a first configuration in which the valve assembly inhibits fluid flow in a direction from the fluid collection bag towards the catheter and permits fluid flow in a direction from the catheter towards the fluid collection bag; and a second configuration in which the valve assembly permits fluid flow in a direction from the fluid collection bag towards the catheter and inhibits fluid flow in a direction from the catheter towards the fluid collection bag.

The method of the seventh aspect of the invention may be a method of manufacturing the catheter assembly of the first aspect of the invention, which, of course, may include any optional feature outlined above.

The method may comprise providing the valve assembly in the first configuration. Thus, the valve assembly may allow the user to fill the fluid collection bag and use the catheter without first needing to change the configuration of the valve assembly.

According to a eighth aspect of the present invention there is provided a method of manufacturing a catheter assembly comprising the steps of providing a catheter having a proximal end for insertion into the body and a distal end and forming a fluid collection bag, the fluid collection bag being formed by providing two panels, creating a peripheral bond between the two panels, and providing an inlet in the fluid collection bag to receive fluid from the catheter, wherein: the two panels are configured to form a base of the fluid collection bag distal from the inlet; and the fluid collection bag is configured to stand up on the base.

The method of the eighth aspect of the invention may be a method of manufacturing the catheter assembly of the second aspect of the invention, which, of course, may include any optional feature outlined above.

The method may comprise providing a base panel. The method may comprise joining the base panel to the front and/or rear panels of the fluid collection bag. The method may comprise folding the base panel. The base panel may be folded along a line parallel to a base edge of the front and/or rear panels.

The method may comprise providing two side panels. The method may comprise joining each side panel to the front and/or rear panels of the fluid collection bag. The method may comprise joining each side panel to the base panel. The method may comprise folding each side panel. Thus, the interior volume of the fluid collection bag may be further increased.

The method may comprise providing a funnel at the distal end of the catheter. The method may comprise positioning the funnel inside the fluid collection bag. Thus, the funnel may be used to prevent separation of the fluid collection bag and catheter.

According to a ninth aspect of the present invention there is provided a method of forming a catheter assembly comprising the steps of providing a catheter having a proximal end for insertion into the body and a distal end, arranging a fluid collection bag to receive fluid from the distal end of the catheter, enclosing the catheter along the length of the catheter from the proximal end to the distal end with a sleeve, and arranging a fluid reservoir at the proximal end of the catheter, wherein the fluid reservoir is configured to release wetting fluid into the sleeve to wet the catheter and wherein the sleeve is in fluid communication with the fluid collection bag.

The method of the ninth aspect of the invention may be a method of forming the catheter assembly of the third aspect of the invention, which, of course, may include any optional feature outlined above.

According to an tenth aspect of the present invention there is provided a method of forming a packaged catheter assembly comprising the steps of providing a fluid reservoir, a catheter, and a pouch, and arranging the fluid reservoir and catheter within the pouch; wherein the catheter comprises a proximal end for insertion into the body, a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

The method of the tenth aspect of the invention may be a method of forming the packaged catheter assembly of the fourth aspect of the invention, which, of course, may include any optional feature outlined above.

The method may comprise arranging the reservoir adapter at the end of the sleeve. The method may comprise inserting the fluid reservoir into the reservoir adapter so as to block/inhibit the passage of the catheter therethrough. Thus, the catheter is protected by the fluid reservoir/reservoir adapter.

The method may comprise forming a housing around a sachet. The method may comprise attaching the housing to the end of the sleeve using the reservoir adapter.

According to an eleventh aspect of the present invention there is provided a method of manufacturing a catheter assembly the method comprising providing a catheter and a fluid collection bag comprising a retaining element, wherein the retaining element is configured to receive at least part of the catheter to stow the catheter after use.

The method of the eleventh aspect of the invention may be a method of manufacturing the packaged catheter assembly of the fifth aspect of the invention, which, of course, may include any optional feature outlined above.

According to a twelfth aspect of the present invention there is provided a method of manufacturing a packaged catheter assembly comprising the steps of providing a catheter, a fluid reservoir comprising wetting fluid, a fluid collection bag and a pouch divided into first and second compartments; arranging the fluid reservoir and at least a portion of the catheter in the first compartment; connecting the fluid collection bag to the catheter; and arranging the fluid connection bag in the second compartment; wherein the pouch is configured to provide an opening allowing access to at least the second compartment, and through which the catheter may be withdrawn.

The method of the twelfth aspect of the invention may be a method of manufacturing the catheter assembly of the sixth aspect of the invention, which, of course, may include any optional feature outlined above.

The method may comprise providing an internal wall in the pouch. The internal wall may divide the pouch into the first and second compartments. The method may comprise sealing a separating edge of the internal wall to a wall (e.g. front or rear) of the pouch. The method may comprise providing a cut-away region in the separating edge. The method may comprise arranging the catheter in the cut-away region.

According to a thirteenth aspect of the present invention there is provided a method of draining a fluid collection bag of a catheter assembly, the catheter assembly comprising a catheter having a proximal end for insertion into the body and a distal end, a valve assembly, and a fluid collection bag arranged to receive fluid from the distal end of the catheter via the valve assembly, the method comprising switching the valve assembly from a first configuration in which the valve assembly inhibits fluid flow in a direction from the fluid collection bag towards the catheter and permits fluid flow in a direction from the catheter towards the fluid collection bag to a second configuration in which the valve assembly permits fluid flow in a direction from the fluid collection bag towards the catheter and inhibits fluid flow in a direction from the catheter towards the fluid collection bag, and allowing the fluid collection bag to drain through the valve assembly.

The method of thirteenth aspect of the present invention may be a method of preparing the catheter assembly of the first aspect of the invention, which, of course, may include any optional feature outlined above and may be manufactured according to the seventh aspect of the invention.

The method may comprise draining the fluid collection bag through the catheter. The method may comprise draining the fluid collection bag through a sleeve configured to enclose the catheter. Thus, the sleeve and catheter may be used to direct the flow of liquid out of the fluid collection bag.

The method may comprise introducing the catheter by its proximal end into the urethra before draining the fluid collection bag. The method may comprise allowing fluid to pass from the body and into the fluid collection bag via the catheter before draining the fluid collection bag. Consequently, the method may be a method of using a catheter assembly.

According to a fourteenth aspect of the present invention there is provided a method of preparing a catheter assembly for use, the catheter assembly comprising a catheter and the fluid collection bag, the fluid collection bag comprising two panels, a peripheral bond between the two panels, and an inlet to receive fluid from the catheter, the method comprising using the two panels to form a base of the fluid collection bag distal from the inlet; and standing the fluid collection bag up on the base.

The method of fourteenth aspect of the present invention may be a method of preparing the catheter assembly of the second aspect of the invention, which, of course, may include any optional feature outlined above and may be manufactured according to the eighth aspect of the invention.

The method may comprise separating the two panels (i.e. the front and rear panels) to form the base. The method may comprise unfolding a base panel of the fluid collection bag. The method may comprise introducing liquid into the fluid collection bag. The method may comprise introducing liquid into the fluid collection bag to automatically form the base in the fluid collection bag.

The method may comprise introducing the catheter by its proximal end into the urethra. The method may comprise allowing fluid to pass from the body and into the fluid collection bag via the catheter. Consequently, the method may be a method of using a catheter assembly.

According to an fifteenth aspect of the present invention there is provided a method of wetting a catheter having a proximal end for insertion into the body and a distal end, the method comprising the steps of releasing wetting fluid from a fluid reservoir at the proximal end of the catheter, into a sleeve that encloses the catheter, allowing the wetting fluid to flow through the sleeve from the proximal end to the distal end, and collecting excess wetting fluid in a fluid collection bag configured to receive fluid from a distal end of the catheter and the sleeve.

The method of the fifteenth aspect of the invention may be a method of wetting the catheter of the catheter assembly of the third aspect of the invention, which, of course, may include any optional feature outlined above and may be manufactured according to the ninth aspect of the invention.

The method may comprise introducing the catheter by its proximal end into the urethra. The method may comprise allowing fluid to pass from the body and into the fluid collection bag via the catheter and/or the sleeve. Consequently, the method may be a method of using a catheter assembly.

According to a sixteenth aspect of the present invention, there is provided a method of unpacking a packaged catheter assembly, the packaged catheter assembly comprising a fluid reservoir, a catheter, and a pouch containing the fluid reservoir and catheter, wherein the catheter comprises a proximal end for insertion into the body, a distal end, and a sleeve extending from the proximal end to the distal end, wherein an end of the sleeve comprises a reservoir adapter configured to engage the fluid reservoir, the method comprising the steps of releasing wetting fluid from the fluid reservoir into the sleeve, and detaching the fluid reservoir to permit the catheter to pass through the reservoir adapter.

The method of the sixteenth aspect of the invention may be a method of wetting a catheter from the packaged catheter assembly of the fourth aspect of the invention, which, of course, may include any optional feature outlined above and may be manufactured according to the tenth aspect of the invention.

The method may comprise forming an opening in the pouch. The step of releasing wetting fluid may comprise rupturing the fluid reservoir, for example by pressing the wetting marker. The method may comprise allowing fluid to flow through the reservoir adapter. Thus, the fluid reservoir may be easily activated to release wetting fluid into the sleeve.

The fluid reservoir may be detached from the reservoir adapter after the release of wetting fluid from the fluid reservoir. Thus, the catheter is adequately wetted before the fluid reservoir is removed.

The method may comprise withdrawing the catheter from the sleeve. Only part of the catheter, preferably including the proximal end of the catheter, may be withdrawn from the sleeve. The method may comprise passing the catheter through the reservoir adapter. The method may comprise bunching the sleeve. The catheter may be withdrawn from the sleeve after it has been wetted. The catheter may be withdrawn from the sleeve after the fluid reservoir has been removed from the sleeve. Thus, the fluid reservoir protects the catheter before use, and the catheter is easily withdrawn through the reservoir adapter.

The method may comprise introducing the catheter by its proximal end into the urethra. The method may comprise allowing fluid to pass from the body and into the fluid collection bag via the catheter and/or the sleeve. Consequently, the method may be a method of using a catheter assembly.

According to a seventeenth aspect of the present invention there is provided a method of stowing a catheter assembly after use, the catheter assembly comprising a catheter and a fluid collection bag comprising a retaining element, the method comprising using the retaining element to receive at least part of the catheter and stow the catheter after use.

The method of the seventeenth aspect of the invention may be a method of stowing a catheter assembly of the fifth aspect of the invention, which, of course, may include any optional feature outlined above and may be manufactured according to the eleventh aspect of the invention.

The method may comprise coiling the catheter in order to stow it. The method may comprise arranging the catheter against the fluid collection bag. The method may comprise retaining the catheter against the fluid collection bag and using the retaining element.

The method may comprise folding an upper region of the fluid collection bag. The method may comprise retaining the fluid collection bag in a folded configuration using the retaining element.

The method may comprise introducing the catheter by its proximal end into the urethra. The method may comprise allowing fluid to pass out of the body via the catheter. Consequently, the method may be a method of using a catheter assembly.

According to an eighteenth aspect of the present invention there is provided a method of unpackaging a packaged catheter assembly, the packaged catheter assembly comprising a catheter, a fluid reservoir comprising wetting fluid, and a pouch, wherein: the pouch is divided into first and second compartments; the fluid reservoir and at least a portion of the catheter are arranged in the first compartment; and a fluid collection bag is connected to the catheter and arranged in the second compartment; the method comprising releasing the wetting fluid into the first compartment so as to wet the catheter arranged therein; forming an opening in the pouch allowing access to at least the second compartment and removing the catheter from the pouch, optionally via the second compartment.

The method of the eighteenth aspect of the invention may be a method of unpackaging a packaged catheter assembly of the sixth aspect of the invention, which, of course, may include any optional feature outlined above and may be manufactured according to the twelfth aspect of the invention.

The method may comprise removing the fluid collection bag from the pouch before removing the catheter.

The method may comprise rupturing/bursting/opening the fluid reservoir within the pouch.

The method may comprise locating the fluid reservoir within the pouch via a window in the pouch and/or a wetting marker disposed on the pouch.

The method may comprise introducing the catheter by its proximal end into the urethra. The method may comprise allowing fluid to pass out of the body via the catheter. Consequently, the method may be a method of using a packaged catheter assembly.

The methods of the fifth to twelfth aspects of the present invention may of course individually include any one or more of the features, optional or otherwise, of one another and further may include any one or more of the features, optional or otherwise, of the first to fourth aspects of the present invention.

In any aspect, a catheter assembly is preferably a closed catheter assembly, that is that the catheter assembly comprises a catheter coupled to a fluid collection bag, such that it is configured to collect and retain fluid released from the body through the catheter. The fluid collected/catheter assembly may then be disposed of by the user when is convenient. Of course, embodiments of the invention may also be open catheter assemblies in that fluid is not collected or retained by the catheter assembly.

### DETAILED DESCRIPTION OF THE INVENTION

In order that the invention may be more clearly understood one or more embodiments thereof will now be described, by way of example only, with reference to the accompanying drawings, of which:

FIG. 1 is a front view of a first embodiment of a packaged catheter assembly where the pouch is in the closed configuration;

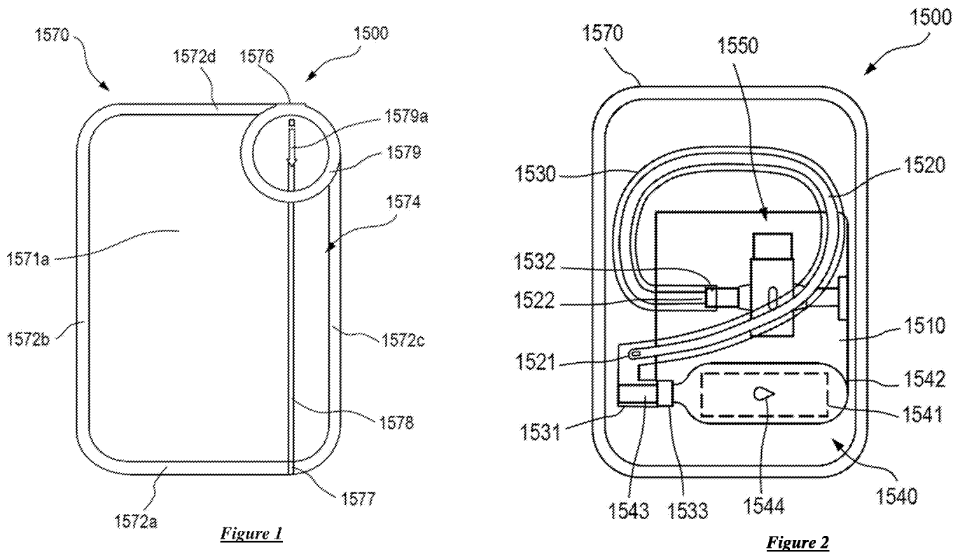

FIG. 2 is a front cut-away view of the packaged catheter assembly of FIG. 1;

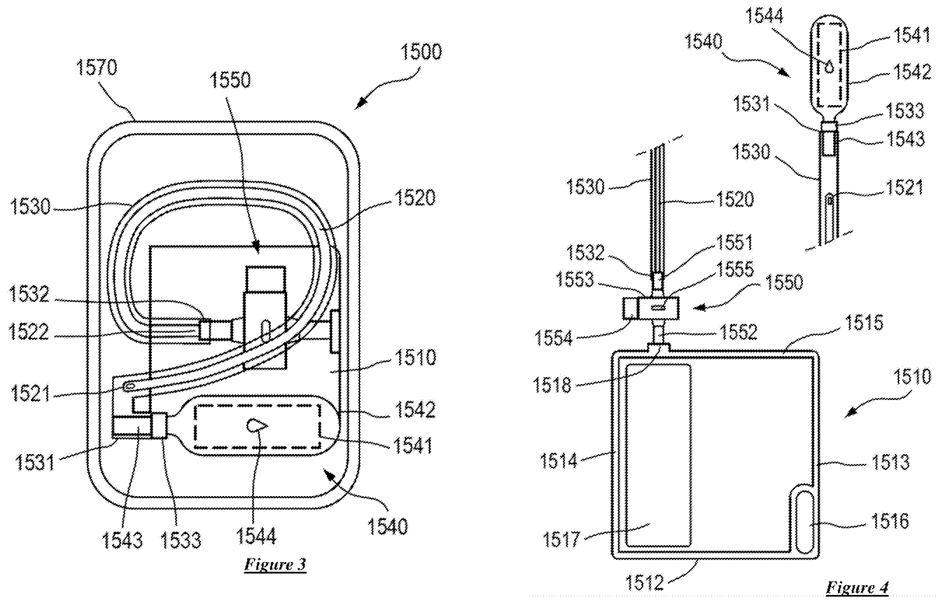

FIG. 3 is a front view of the packaged catheter assembly of FIG. 1 where the pouch is in the open configuration and the catheter assembly contained within the pouch is being removed from the pouch;

FIG. 4 is a front view of the catheter assembly from the packaged catheter assembly of FIG. 1;

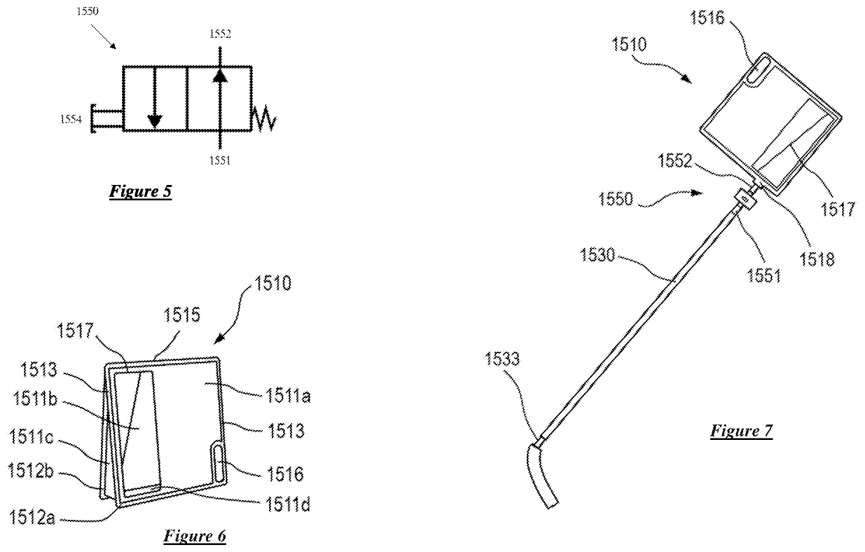

FIG. 5 is a schematic diagram of showing the operation of the valve assembly of the packaged catheter assembly of FIG. 1;

FIG. 6 is a perspective view of the fluid collection bag of the packaged catheter assembly of FIG. 1;

FIG. 7 is a front view of liquid being drained from the fluid collection bag of the packaged catheter assembly of FIG. 1;

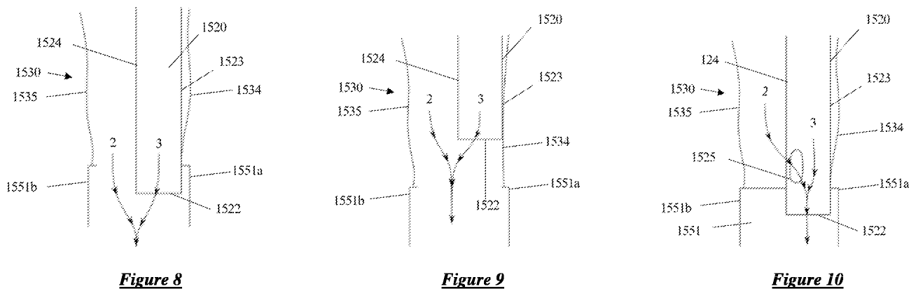

FIG. 8 is a cut-away view of a first example of the inlet of the catheter assembly of FIG. 1;

FIG. 9 is a cut-away view of a second example of the inlet of the catheter assembly of FIG. 1;

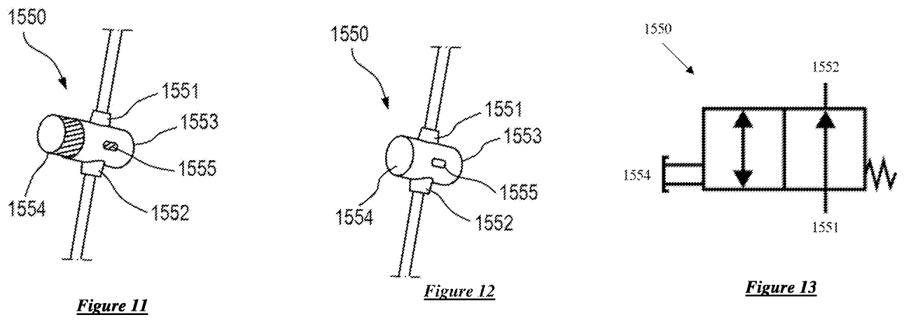

FIG. 10 is a cut-away view of a third example of the inlet of the catheter assembly of FIG. 1;

FIG. 11 is a perspective view of a variation on the valve assembly of the packaged catheter assembly of FIG. 1 in the first configuration;

FIG. 12 is a perspective view of the valve assembly of FIG. 11 in the second configuration;

FIG. 13 is a schematic diagram of showing the operation of the valve assembly of FIG. 11;

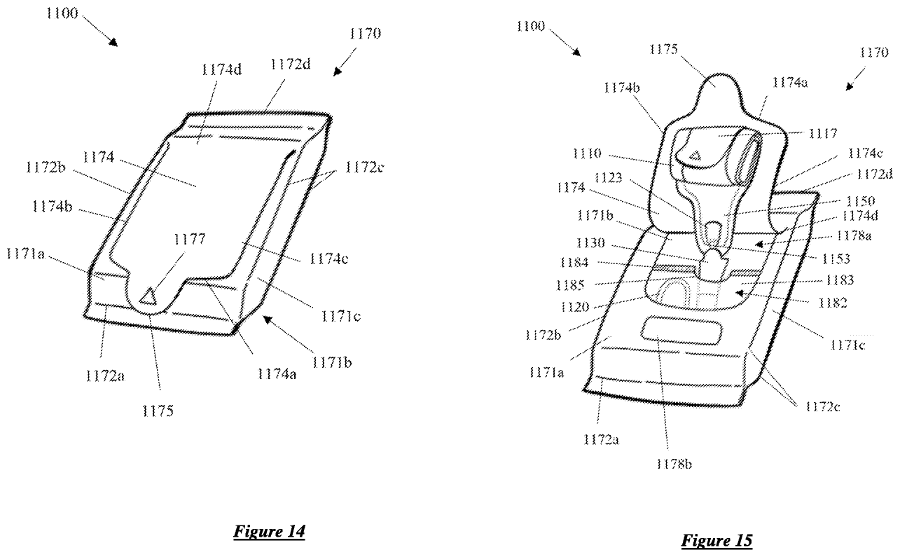

FIG. 14 is a perspective view of a second embodiment of a packaged catheter assembly where the pouch is in the closed configuration;

FIG. 15 is a perspective view of the packaged catheter assembly of FIG. 8 where the pouch is in the open configuration;

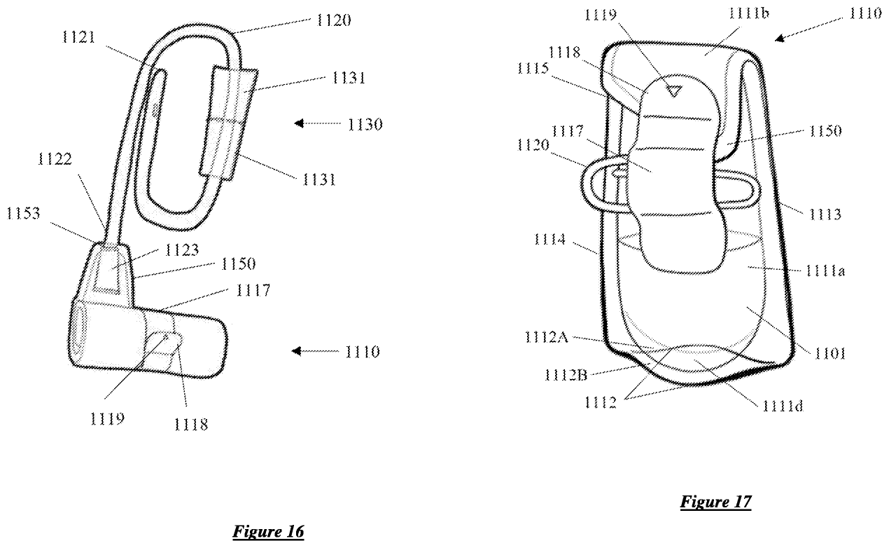

FIG. 16 is a front view of the catheter and fluid collection bag of the packaged catheter assembly of FIG. 8; and

FIG. 17 is a front perspective view of the fluid collection bag of the packaged catheter assembly of FIG. 8 after use.

Referring to FIGS. 1-10, a first embodiment of a packaged catheter assembly 1500 is shown. In the Figures, as is conventional, dashed lines indicate hidden features.

The packaged catheter assembly 1500 comprises a catheter assembly contained within a pouch 1570. The catheter assembly comprises a fluid collection bag 1510, a catheter 1520, a sleeve 1530 and a fluid reservoir 1540. The catheter 1520 has a proximal end 1521 for insertion into the body and a distal end 1522, and the fluid collection bag 1510 is arranged to receive fluid from the distal end 1522 of the catheter 1520, as such the catheter assembly is a “closed catheter assembly”.

In this embodiment, the catheter 1520 is a male urinary catheter made from a hydrophilic thermoplastic elastomer (TPE). The sleeve 1530 of this embodiment is a thermoplastic polyurethane (TPU) or low-density polyethylene (LDPE). Obviously those skilled in the art will be able to select suitable alternative examples.

The pouch 1570 is formed from a front wall 1571a and rear wall 1571b of identical shape and size, and a peripheral seal joining the periphery of the walls to form the pouch 1571 and containing its contents (i.e. the catheter assembly).

The peripheral seal defines a base 1572a, a left lateral edge 1572b, a right lateral edge 1572c and an upper edge 1572d of the pouch 1570. The left lateral edge 1572b and right lateral edge 1572c being defined as the left and right sides of the pouch 1500 when viewing the pouch 1570 with the rear wall 1571b behind the front wall 1571a, the base 1572a at the bottom of the pouch 1570 and the upper edge 1572d at the top of the pouch 1570. The peripheral seal thus defines a pouch 1570 that is generally rectangular with rounded corners and suitably has a width between the lateral edges of between 60-110 mm, preferably 90-110 mm, for example about 105 mm, and a height from the base 1572a to the upper edge 1572d of between 100 to 250 mm, preferably 120 to 170 mm, for example about 150 mm. The base 1572a defines the bottom of the pouch 1570 in use, and the upper edge 1572d the top. However, in other embodiments alternative shapes and sizes could be conceived, such as elliptical or circular walls forming correspondingly shaped pouches, or even a tubular pouch or pouch with more than two walls, e.g. a prism or other polygon.

The peripheral seal may be formed from any suitable bonding/sealing methods such as chemical adhesives, chemical bonding, ultrasonic bonding, or mechanical bonding. In alternative embodiments, the pouch 1570 may be formed from a single piece of material. The pouch may then be formed by folding this single piece of material along a pouch fold line to form the walls. In addition, in some embodiments any number of folds may be used to form the pouch 1570. For example, none of the edges may comprise a fold (as in the illustrated example), one edge may comprise a fold, or two or more of the edges may comprise a fold.

In this embodiment, the pouch 1570 is formed from an opaque material and as such provides a discrete package for its contents which can make the user more comfortable in carrying the packaged catheter assembly 1500 in day-to-day life. In addition, the exterior of the pouch 1570 may have a textured or printed appearance, for example a matte appearance, and feel to reduce the impression that it is a medical device.

In this embodiment, the pouch 1570 also comprises an interaction region which is arranged adjacent to the right lateral edge 1572c of the pouch 1570 on the front wall 1571a. The interaction region is used to provide access to the pouch through/near to the right lateral edge 1572c. In this embodiment, the interaction region comprises a rectangular break-away region 1574. The break-away region 1574 comprises a tear start 1576 at the point the upper edge 1572d meets the break-away region 1575, a tear stop 1577 at a corresponding point on the base 1572a, and a tear line 1578 spanning between the tear start 1576 and tear stop 1577. The tear line 1578 comprises a line of weakness in the walls of the pouch 1570 configured to preferentially tear and allow the break-away region to be at least partially separated from the pouch 1570. The tear line 1578 may be formed by any suitable means but in this embodiment is laser etched.

In this embodiment, the tear line 1578 is positioned off-centre with respect to the width of the pouch 1570. The tear line 1578 is positioned 70-90% across the width of the pouch 1570 from the left lateral edge 1572b to the right lateral edge 1572c, for example 80% across. In other embodiments, the tear-away region may have a different shape or size as required by the shape/size of the pouch 1570 or the contents of the pouch 1570. For example, it may be positioned centrally with respect to the width and/or may be arranged in a different orientation such that access is provided through a corner of the pouch 1570 or a different edge of the pouch 1570.

In this embodiment, the tear line 1578 extends across the front wall 1571a only and not the rear wall 1571b. As such, once the tear line 1578 is torn, the break-away region 1574 remains attached to the pouch 1570 by the rear wall 1571b. This makes the pouch 1570 more convenient to use as the break-away region doesn't become a separate item and so the user doesn't need to handle/store/dispose of an extra item once the tear line 1578 is activated. In other embodiments, the break-away region 1574 may be configured to completely separate from the pouch 1570. This could be advantageous to allow easier access to the pouch 1570.

In this embodiment, the break-away region 1574 comprises a pull-ring 1579 arranged adjacent to the tear start 1576. The pull ring 1579 is sized to allow a finger to pass through and grip then pull so as to tear the front wall 1571a along the tear line 1578 and create an opening in the pouch 1570. Of course, in other embodiments the pull ring 1579 may be replaced by a different kind of easily gripped object, such as a tab.

In this embodiment, the pull-ring 1579 is a different colour from the front wall 1571 of the pouch 1570 to allow the user to more easily recognise it and open the pouch 1570. In addition, an opening marker 1579a in the form of an arrow is present on the front wall 1517a in a position corresponding to the pull-ring 1579 and tear line 1578 and pointing the towards the base 1572a of the pouch 1570. The opening marker 1579a is the same colour as the pull-ring 1579. The user is therefore guided to open the pouch 1570. Of course, in other embodiments, the pull-ring may be a different shade of colour from the pouch, or may be the same colour/shade of colour where discretion is prioritised. In addition, the opening marker may not be present or may have a different form. In some embodiments, it may comprise a region which is a different texture to the pouch for example where the user is visually impaired.

In this embodiment, the fluid reservoir 1540 is configured to retain a wetting fluid and release wetting fluid to activate the surface of the catheter 1520. In this embodiment, the wetting fluid is water and interacts with the hydrophilic surface of the catheter 1520 to render it lubricious. In other embodiments, other wetting fluids may be used and they may be polar (e.g. water-based) or non-polar (e.g. oil-based) depending on the catheter's surface properties. In this embodiment, the fluid reservoir 1540 is configured to retain and release 12 ml of wetting fluid, of course in other embodiments more or less wetting fluid may be required.

In this embodiment, the fluid reservoir 1540 comprises a burstable rectangular sachet 1541 configured to retain 5 to 50 ml of wetting fluid, for example 12 ml. In this embodiment, the fluid reservoir also comprises a housing 1542 that surrounds the sachet 1541. The housing 1542 comprises a flexible and fluid impermeable material such as soft silicone. In other embodiments, the housing 1542 may comprise a thermoplastic polyurethane (TPU) or low-density polyethylene (LDPE). The housing 1542 is configured to enclose the sachet 1415 on all sides and is tight fitting on all sides, as such the housing 1542 is also generally rectangular in shape but with rounded edges.

In this embodiment, the housing 1542 is push-fit into a wetting tube 1543 at one end that extends away from the fluid reservoir 1540, of course in other embodiments screw fittings or seals such as a weld may join the housing 1542 to the wetting tube 1543. In this embodiment, the housing 1542 has an aspect ratio (length:width:height) of 5:2:2 and as such is elongate. The wetting tube 1543 connects to a middle of a face of the housing 1542 that is perpendicular to the length of the housing 1542. In this embodiment, the wetting tube 1543 extends in a direction parallel to the length of the housing 1542 and is approximately one third the length of the housing 1542 with a circular cross-section and a diameter approximately half the width and/or height of the housing 1542. The face of the housing 1542 on the opposite side from the wetting tube 1543 is rounded to the extent that it forms a hemispherical end of the housing 1542.

The user can release wetting fluid held by the fluid reservoir 1540 by compressing the fluid reservoir 1540, for example by pinching the housing 1452 between their fingers. To facilitate the user in activating the fluid reservoir 1540, the housing 1542 comprises a wetting marker 1544. The wetting marker 1544 comprises a region of the housing 1542 that is a different colour or texture to the rest of the housing 1542 to allow the user to easily identify the position of the sachet 1451 and where to press to activate it and release wetting fluid. In other embodiments, the fluid reservoir 1540 may be configured to release wetting fluid through a different means, for example by twisting or bursting the fluid reservoir in a different way.

The sleeve 1530 of this embodiment is sized to enclose the catheter 1520 along the majority of the length of the catheter 1520, and preferably the entire length of the catheter 1520. The sleeve 1530 comprises a proximal end 1531 corresponding to the proximal end 1521 of the catheter 1520 and a distal end 1532 corresponding to the distal end 1522 of the catheter 1520. The distal end 1532 of the sleeve 1530 is attached to a valve assembly 1550 as described below. The proximal end 1531 of the sleeve is configured to receive and engage the wetting tube 1543 such that wetting fluid released by the fluid reservoir 1540 is directed into the sleeve 1530 as described further below.

In this embodiment, the proximal end 1531 of the sleeve 1530 comprises a reservoir adapter 1533 configured to retain the fluid reservoir 1540 at the proximal end 1531 of the sleeve 1530. In this embodiment, the reservoir adapter 1533 achieves this by retaining the wetting tube 1543 and housing 1542 in engagement with the sleeve 1530. The reservoir adapter 1533 is fitted around the sleeve 1530 and wetting tube 1543 and comprises an O-ring with an inner diameter that is slightly less than the outer diameter of the wetting tube 1543. Consequently, when the reservoir adapter 1533 is fitted around the wetting tube 1543 and proximal end 1531 of the sleeve 1530, the reservoir adapter 1533 urges the sleeve 1530 against the wetting tube 1543. Thus, a fluid-tight seal is formed between the sleeve 1530 and wetting tube 1543 and the fluid reservoir 1540 is retained in position via the housing 1542.

To ensure tight engagement between the wetting tube 1543 and sleeve 1530, the sleeve 1530 should be formed from a material that is more flexible than both the wetting tube 1543 and reservoir adapter 1533. Preferably, the reservoir adapter 1533 is more flexible than the wetting tube 1543 to ensure the wetting tube 1543 is not compressed when engaged by the reservoir adapter 1533. If the wetting tube 1543 is compressed, this could lead to an unwanted reduction or restriction of wetting fluid flow from the fluid reservoir 1540 to the sleeve 1530. In this embodiment, the reservoir adapter 1533 comprises rubber, however, in other embodiments, a different material may be used such as silicone.

In this embodiment, the reservoir adapter 1533 is configured to release the fluid reservoir 1540 and housing 1542 to allow the proximal end 1521 of the catheter 1520 to pass out of the sleeve 1530 and be used. In this embodiment, the housing 1542 can be released from the reservoir adapter 1533 by being simply pulled out of the sleeve 1530. In other embodiments, specific fittings or tear-lines may be used to allow the housing 1542 to be released from the reservoir adapter 1533. In this embodiment, the wetting tube 1543 is not part of the fluid reservoir 1540 and thus, when the housing 1542 is removed from the sleeve 1530, the wetting tube 1543 remains in the proximal end of the sleeve 1530 to be used as a handling element. In other embodiments, the wetting tube 1543 may be integrally formed with the housing 1542 and may be removed from the sleeve 1530 with the housing 1542.

In this embodiment, the valve assembly 1550 is connected to the distal end 1532 of the sleeve 1530 and the fluid collection bag 1510 and is configured to control the flow of fluids between them. In this embodiment, the valve assembly 1550 comprises two configurations: a first configuration in which the valve assembly 1550 inhibits fluid flow in a direction from the fluid collection bag 1510 towards the catheter 1520 and permits fluid flow in a direction from the catheter 1520 towards the fluid collection bag 1510; and a second configuration in which the valve assembly 1550 permits fluid flow in a direction from the fluid collection bag 1510 towards the catheter 1520 and inhibits fluid flow in a direction from the catheter 1520 towards the fluid collection bag 1510. Thus, the valve assembly 1550 allows the user to both fill and empty the fluid collection bag 1510 via the sleeve 1530 in a controlled manner as described below.

In this embodiment, the valve assembly 1550 comprises a proximal passageway 1551 configured to provide a fluid communication between the valve assembly 1550 and the distal end 1532 of the sleeve 1530 and the catheter 1420, and a distal passageway 1552 configured to provide a fluid connection between the valve assembly 1550 and the fluid collection bag 1510. The connection between the sleeve 1530 and/or catheter 1520 and the proximal passageway 1551 is flexible as described further below. Each passageway 1551, 1552 connects to opposite sides of a side wall of a cylindrical valve housing 1553 and the valve assembly 1550 is configured to control the fluid flow between the passageways 1551, 1552 as shown in FIG. 5 and described below. The valve housing 1553 has a diameter of approximately 1 to 3 cm, for example 2 cm and a length approximately double its diameter. In this embodiment, each passageway 1551, 1552 extends away from the valve housing 1553 by a distance that is not more than the diameter of the valve housing 1553. Each passageway 1551, 1552 is tubular with an internal diameter that is approximately equal to the internal diameter of the catheter 1520, thus each passageway 1551, 1552 has a similar fluid resistance to the catheter 1520 and does not significantly inhibit the flow of fluid into/out of the fluid collection bag 1510.

In this embodiment, one end of the valve housing 1553 is open and is filled by a user operable control which comprises a button 1554 of cylindrical shape. The button 1554 is configured to allow the user to select the first or second configurations of the valve assembly 1550 and may be depressed in an axial direction of the valve housing 1553. In one example, the button 1554 may be in an extended configuration when the valve assembly 1550 is in the first configuration and a depressed configuration when the valve assembly 1550 is in the second configuration. In the extended configuration, the button 1554 extends beyond the open end of the valve housing 1553 and in the depressed configuration, the button 1554 is flush with the open end of the valve housing 1553. The user can switch between the configurations by depressing the button 1554 past the depressed configuration, for example an audible/tactile click may signal that the button 1554 has been sufficiently depressed, for example via a protrusion (not shown) on the inside of the valve housing 1553 that overrides a detent (not shown) on the button 1554 as the button 1554 moves with respect to the valve housing 1553. The user operable control also comprises a biasing element in the form of a spring (not shown) to bias the user operable control and ensure that the button 154 is always returned to the correct extended configuration. Thus, the valve assembly 1550 may be operated using a simple button and can be readily operated by someone with reduced dexterity by simply squeezing the button in their hand. Of course, other configurations of the button 1554 may be readily implemented by the skilled person or indeed a different type of user operable control may be used such as a switch, button, lever, slider, knob. Thus, the valve can be flexibly operated as required by the user.

In this embodiment, the valve housing 1554 comprises a window 1555 located in the side wall of the valve housing 1554 between the passageways 1551, 1552. The window 1555 may be configured to indicate to the user if the valve assembly 1550 is in the first or second configuration as described further below. The window 1555 is elongate with a length parallel to the axis of the valve housing 1554. The window 1555 has a length of approximately half the diameter of the valve housing 1554 and the window has an aspect ratio (length:width) of approximately 3:1. The window is stadium-shaped that is rectangular semicircles replacing two opposite sides.

In this embodiment, the side wall of the button 1554 is a different colour from the valve housing 1553 and a top end of the button 1554 (that is the end that is outside the valve housing 1553) comprises an activation marker 1556. In this embodiment, the activation marker 1556 comprises the words “PUSH” to provide visual guidance on how to operate the valve assembly 1550. In other embodiments, the side wall of the button may be a different shade of colour from the valve housing, and/or the entire button may be a different colour/shade of colour from the valve housing. This can help the user to identify the button 1554 and use the valve assembly 1550.

In addition, the button 1554 is visible through the window 1555 described above. In this embodiment, the parts of the button 1554 that are visible through the window 1555 when the button 1554 is in the extended and depressed configurations respectively are different colours or different shades of colour. This helps the user to identify which configuration the valve assembly 1550 is in. In this embodiment, the window 1555 appears to be the same colour as the button 1554 when the button 1554 is in the extended configuration and is a different colour from the button 1554 when the button 1554 is in the depressed configuration. The user is thereby given a visual indication of the state of the valve assembly 1550.

Inside the valve housing 1553 and between the passageways 1551, 1552, there is a valve body (not shown) that is configured enable the valve assembly 1550 to operate as shown in FIG. 5. The valve body is configured to inhibit fluid flow in a direction from the fluid collection bag 1510 and distal passageway 1552 towards the catheter 1520 and proximal passageway 1551 and permit fluid flow in a direction from the catheter 1520 and proximal passageway 1551 towards the fluid collection bag 1510 and distal passageway 1552 when the button 1554 is in the extended configuration. The valve body is also configured to permit fluid flow in a direction from the fluid collection bag 1510 towards the catheter 1520 and inhibits fluid flow in a direction from the catheter 1520 towards the fluid collection bag 1510 and distal passageway 1552 when the button 1554 is in the depressed configuration. Thus, the valve body enables the valve assembly 1550 to function as required.

The valve body could be any suitable arrangement of fluidic components as would be familiar to the skilled person to enable the valve assembly 1550 to operate as described above. In one example, the valve body comprises two opposing check valves in series: a first check valve to prevent fluid flow in a direction from the catheter 1520 and proximal passageway 1551 towards the fluid collection bag 1510 and distal passageway 1552, and a second check valve to prevent fluid flow in a direction from the fluid collection bag 1510 and distal passageway 1552 towards the catheter 1520/proximal passageway 1551, for example spring loaded in-line check valves could be used for the first and second check valves. The button 1554 is then configured to inhibit the operation of the second check valve in the extended configuration and the first check valve when in the depressed configuration, for example by physically preventing the respective check valve from closing. In a different example, the valve body may comprise two opposing check valves as described above but stacked on top of one another in parallel within the valve housing and coupled to the button 1554. Movement of the button 1554 between the extended and depressed configurations may then select which check valve is in fluid communication with the passageways 1551, 1552 and thus alter the polarity of the valve assembly 1550. Thus, the button 1554 and valve body provide the first and second configurations of the valve assembly 1550. Of course, in other embodiments, a lever, switch or other user operable input may be used to control the valve assembly 1550 and a different type of valve body may be used to achieve the same effect.

In this embodiment, the distal passageway 1552 is connected directly to an access opening 1518 of the fluid collection bag 1510, that is without any intervening components such as tubing. The access opening 1518 is configured to allow fluid to flow into and out of the fluid collection bag 1510 as required. In other embodiments, the distal passageway may be connected indirectly to the access opening of the fluid collection bag 1510a, for example via an intervening section of tubing or other fluid connection device placed between the distal passageway and fluid collection bag.

In this embodiment, the fluid collection bag 1510 comprises a front panel 1511a, a rear panel 1511b of identical shape and size, and a peripheral bond joining the periphery of the panels to form the bag 1510. The peripheral bond defines a base 1512, a right lateral edge 1513, a left lateral edge 1514 and an upper edge 1515. The right lateral edge 1513 and left lateral edge 1514 being defined as the right and left sides of the bag 1510 when viewing the bag 1510 with the rear panel behind the front panel 1511, the base 1512 at the bottom of the bag 1510 and the upper edge 1515 at the top of the bag 1510. The peripheral bond thus defines a bag 1510 that is generally rectangular and preferably square with a width between the lateral edges of between 25 and 40 cm, 30 and 35 cm, or most preferably 33 and 35 cm, and a height from the base 1512 to the upper edge 1515 of between 25 and 40 cm, 30 and 35 cm, or most preferably 33 and 35 cm. The bag 1510 is thus configured to hold at least 700 ml of fluid released from the user through the catheter safely. The base 1512 defines the bottom of the bag 1510 in use, and the upper edge 1515 the top. However, in other embodiments alternative shapes and sizes could be conceived, such as elliptical or circular panels forming correspondingly shaped bags, or even a tubular bag or bag with more than two panels, e.g. a prism or other polygon.

In this embodiment, the fluid collection bag comprises two side panels 1511c and a base panel 1511d. The side panels 1511c are each triangular shaped in the form of elongate isosceles triangles with a height equal to the height of the fluid collection bag 1510. The two equal length sides of each side panel 1511c are bonded to the front and rear panels 1511a, 1511b respectively with one side panel 1511c separating the front and rear panels 1511a, 1511b along the left lateral edge 1514 of the fluid collection bag 1510 and the other separating the front and rear panels 1511a, 1511b along the right lateral edge 1513.

The base panel 1511d that spans between the front panel 1511a and rear panel 1511b along the base 1512 of the peripheral bond and between the base edges of the side panels 1511c. The base panel 1511d is therefore rectangular shaped with a width equal to the width of the fluid collection bag 1510. The base panel 1511d has a length that is equal to the length of the base edge of the side panels 1511c and the base panel 1511d has an aspect ratio (length:width) of approximately 1:4. The base panel 1511d and side panels 1511c thus allow the front panel 1511a and rear panel 1511b to separate along the base 1512 and form a wedge-shaped fluid collection bag 1510.

Consequently, the base panel 1511d and side panels 1511c allow the fluid collection bag 1510 to have a greater internal volume and also to be free-standing when partially filled with fluid as described below.

The base 1512 is formed by two flanges 1512a, 1512b; the rear panel joined to the base panel by the peripheral bond forms a first flange 1512a and the front panel joined to the base panel by the peripheral bond forms the second flange 1512b. The two flanges 1512a, 1512a extend around the left lateral edge 1514, base 1512 and right lateral edge 1513 of the fluid collection bag 1510 where the side panels 1511c and base panel 1511d meets the rear panel 1511b and the front panel 1511a respectively.

In this embodiment, the panels comprise a flexible plastics material, for example polypropylene (PP), polyethylene terephthalate (PET), low density polyethylene (LDPE), metalized polyester (MET PET) orientated polypropylene (OPP), polyvinylpyrrolidone (PVP), or polyvinyl chloride (PVC). Consequently, the panels 1511a, 1511b, 1511c, 1511d may be folded or rolled to reduce the physical size of the fluid collection bag 1510 before, during or after use. In this embodiment, the fluid collection bag 1510 is provided in a stowed configuration before use, which is a folded configuration as described in more detail below.

In this embodiment, the corner of the peripheral bond where the right lateral edge 1514 and base 1512 meet comprises a finger hole 1516 to allow the bag 1510 to be easily handled by a user. The finger hole 1516 is stadium shaped, that is a rectangle with semicircles replacing two opposite sides. The finger hole 1516 comprises two elongate straight edges with a length of one quarter to one third, for example 30%, of the height of the fluid collection bag 1510 and aligned parallel to the right lateral edge 1514. The finger hole 1516 has an aspect ratio (length:width) of approximately 4:1. In other embodiments, the finger hole may comprise a series of ridges, or otherwise easily gripped surface or feature to allow the user to easily handle the bag. In addition, there may be different numbers of finger holes, such as two or more, and they may be placed in different locations on the bag.

In this embodiment, the panels 1511a, 1511b, 1511c, 1511d are formed from an opaque material. This makes the fluid collection bag 1510 more discreet for the user. Of course, in other embodiments, any one or more of the panels may be transparent or translucent.

In this embodiment, the fluid collection bag 1510 further comprises a fill level indicator 1517. The fill level indicator 1517 comprises a region of the front panel 1511a that is formed from a transparent or translucent material configured to allow a user to visualise the amount of liquid within the fluid collection bag 1510. The fill level indicator spans a majority of the height of the fluid collection bag 1510 and in this embodiment spans over 90% of the height of the fluid collection bag 1510. In this embodiment, the fill level indicator 1517 has a width of approximately 30-50% the width of the fluid collection bag 1510, for example 35-40% or most preferably 37%. Thus, the fill level indicator 1517 is a rectangular window that can be used to visualise the contents of the fluid collection bag 1510.

In this embodiment, the fill level indicator 1517 does not contain any fill level markers to allow the fill level of the fluid collection bag 1510 to be quantitatively measured. Of course, in other embodiments, fill level markers may be present for example to let the user identify when the fluid collection bag 1510 is a certain % full, for example the fill level markers may indicate 25%, 50%, and 75% fill levels of the fluid collection bag.

Referring to FIG. 7, in this embodiment, the finger hole 1516, fill level indicator 1517 and access opening 1518 are positioned with respect to one another to enhance the usability of the fluid collection bag 1510.

In this embodiment, the access opening 1518 is positioned in the upper edge 1515 of the fluid collection bag 1510 at a point approximately 15-30% along the upper edge 1515 from the left lateral edge 1514 to the right lateral edge 1513. Thus, the access opening 1518 is positioned at or near an opposite corner of the fluid collection bag 1510 from the finger hole 1516. This allows the user to easily grasp the finger hole 1516 and allow the fluid collection bag 1510 to drain completely through the access opening 1518 while the bag 1510 hangs from the finger hole 1516 and without further manipulation.

In this embodiment, the fill level indicator 1517 spans the majority of the height of the fluid collection bag 1517 and is positioned adjacent to the left lateral edge 1514 of the fluid collection bag 1517. Thus, the fill level indicator 1517 is on the same side of the fluid collection bag 1517 as the access opening 1518 and ensures that during filling and draining the fluid within the fluid collection bag 1510 is visible as soon as it enters the bag and until it leaves the bag. This helps to ensure the fluid collection bag 1510 is completely emptied when draining the bag.

Referring to FIGS. 2 and 3, in this embodiment, the fluid collection bag 1510 is provided in a stowed folded configuration as mentioned above. The fluid collection bag 1510 is folded once at a mid-point along its width and then again at a midpoint along its height thus in the stowed configuration the fluid collection bag 1510 is a square with a width/height half its unfolded width/height. When contained within the pouch 1570, the access opening 1518 is also folded such that the valve assembly 1550 is positioned to overlie the fluid collection bag 1510 and minimise the physical size of the catheter assembly. The valve assembly 1550 is initially provided in the first configuration and as such permits flow of fluids from the catheter 1520 into the fluid collection bag 1510, in addition, the button 1554 is in the extended configuration.

The position and configuration of the catheter 1520 is not particularly limited so long as it is not curved to the extent that damage to the catheter is likely, for example from kinking. In this embodiment, the catheter 1520 is arranged in a curved/coiled configuration, this helps ensure that the catheter 1520 is not excessively curved and minimises risk of damage.

In this embodiment, the fluid reservoir 1540/housing 1542 is positioned adjacent to the base 1572a of the pouch 1570 and beneath the valve assembly 1550. The wetting tube 1543 is therefore positioned adjacent to the left lateral edge 1572b of the pouch 1570. The sleeve 1530 extends from the wetting tube 1543 and completes a hairpin turn before enclosing the proximal end 1521 of the catheter 1520. This takes advantage of the flexible nature of the sleeve 1530 which can be arranged with tight turns/folds to reduce the size of the catheter assembly. This is because even if the sleeve 1530 becomes kinked, kinks in the sleeve 1530 are unlikely to negatively affect the usability of the catheter assembly. It is of course important that any such tight turns/folds are present in a portion of the sleeve 1530 that does not enclose the catheter 1520 as kinks in the catheter 1520 may render it unusable.

Prior to use, the pouch 1570 is provided sealed closed with the contents arranged within it as mentioned above. The contents of the pouch 1570 can be accessed by opening the pouch 1570. The pouch 1570 is opened by grasping the pouch 1570 in one hand and pulling the pull-ring 1579 in the direction indicated by the opening marker 1579a. This breaks the front wall 1571a of the pouch 1570 along the tear line 1578 from the tear start 1576 in the upper edge 1572d to the tear stop in the base 1572a. Thus, an opening is created and the break-away region 1574 is partially removed from the pouch 1570 allowing the catheter assembly to be removed from the pouch 1570 as shown in FIG. 3.

Once the pouch 1570 has been opened, the fluid collection bag 1510, catheter 1520 and sleeve 1530 may be removed from the pouch 1570. To ensure the catheter 1520 is wetted prior to use, the user must activate the fluid reservoir 1540 by squeezing the housing 1542 at the position indicated by the wetting marker 1544. Preferably, the user visualises the wetting fluid leaving the housing 1542 through the wetting tube 1543 and into the sleeve 1530. The wetting fluid enters the sleeve 1530 and flows down the sleeve 1530 wetting the catheter 1520 from its proximal end 1521 to its distal end 1522. This prioritises wetting of the proximal end 1521 and ensures that it is wetted prior to use which is important to reduce the likelihood of injury during use as the proximal end 1521 is the first part of the catheter 1520 to be introduced into the body.

The wetting fluid may then flow through the valve assembly 1550 and into the fluid collection bag 1510 where it is collected.

Before use, the fluid collection bag 1510 may be unfolded to ensure that the collection of liquid within it is not hindered by folds in the fluid collection bag 1510. In addition, the fluid collection bag 1510 may be opened such that the side panels 1551c and base panel 1511d are unfolded and the fluid collection bag 1510 is free-standing on its base. This can be helpful as the user does not need to hold or support the fluid collection bag 1510 and as the access opening 1518 is on the upper edge 1515 of the fluid collection bag 1510, fluid will efficiently fill the bag while it remains standing upright.

The fluid reservoir 1540 and housing 1542 initially block the catheter 1520 from leaving the sleeve 1530 and as such, the user must then remove the housing 1542 from the sleeve by grasping the reservoir adapter 1533 in one hand and pulling the housing 1542 away from it with the other. The reservoir adapter 1533 then separates from the wetting tube 1543 leaving the wetting tube 1543 in place within the sleeve 1530 and restricted by the reservoir adapter 1533. Once the housing 1542 has been removed, the proximal end 1521 of the catheter 1520 may emerge from the sleeve 1530 and be progressively introduced into the body until urine flows therethrough and into the fluid collection bag 1510 via the valve assembly 1550. In this embodiment, the relatively rigid wetting tube 1543 provides a handling element that can be used to guide the proximal end 1521 of the catheter 1520 into the body without having to touch the catheter 1520 directly and risk infection.

Referring to FIG. 7, after use of the catheter 1520, the user may wish to empty the contents of the fluid collection bag 1510. This can be desirable in order to reduce the size and weight of the used bag 1510 and/or to dispose of the bag 1510. To allow the bag 1510 to be emptied, the valve assembly 1550 must be switched into the second configuration by depression of the button 1554 to the depressed configuration. The fluid collection bag 1510 may also be grasped and lifted by the finger hole 1516 to allow gravity to drive fluid flow out of the access opening 1518, through the valve assembly 1550 and out through the catheter 1520/sleeve 1530. The wetting tube 1543 may once again be used to guide the flow of fluid out of the sleeve 1530 to minimise the risk of spillages.

Advantageously, the valve assembly 1550 may be used to selectively start and stop fluid flow through switching between the first and second configurations using the button 1554. This can allow the user to better control draining to minimise leaks/spillage of fluids, for example, they may hang the fluid collection bag 1510 in an elevated position and arrange the sleeve 1530/catheter 1520 into a receptacle such as a drain or toilet before depressing the button 1554 and allowing fluid to drain out of the fluid collection bag 1510.

As mentioned above, in this embodiment the catheter 1520 and sleeve 1530 are in fluid communication with the proximal passageway 1551. The configuration of the catheter 1520, sleeve 5130, and proximal passageway 1551 is relatively flexible and many different configurations could be employed in embodiments of the invention.

Referring to FIG. 8, in a first example of the configuration for the catheter 1520, sleeve 1530 and proximal passageway 1551 fluid passes directly from the sleeve 1530 to the proximal passageway 1551 and directly from the catheter 1520 to the proximal passageway 1551. In this example, a right lateral side 1523 of the catheter 1520 is attached to a right side 1551a of the proximal passageway 1551 and there is a gap between a left lateral side 1524 of the catheter 1520 and the respective left side 1551b of the proximal passageway 1551. In addition, the distal end 1522 of the catheter 1520 is within the proximal passageway 1551, and the sleeve 1530 is sealed around a periphery of the proximal passageway 1551. As such, fluid travelling within the sleeve 2 is directed into the proximal passageway 1551 directly via the gap between the left lateral side 1524 of the catheter 1520 and the left side 1551b of the proximal passageway 1551. In addition, fluid travelling within the catheter 3 passes out the distal end 1522 and into the proximal passageway 1551 directly. In other examples, the catheter 1520 may be positioned differently and may be attached to the left side 1551b of the proximal passageway 1551, or to a front or rear side.

Referring to FIG. 9, in a second example fluid passes from the catheter 1520 into the sleeve 1530 and then into the proximal passageway 1551. In this example, the right lateral side 1523 of the catheter 1520 is attached to a left side 1534 of the sleeve 1530 and a distal end 1522 of the catheter 1520 is not within the proximal passageway 1551. As in the first example, the sleeve 1530 is sealed around a periphery of the proximal passageway 1551. As such, fluid travelling within the sleeve 2 is directed into the proximal passageway 1551 directly and fluid travelling within the catheter 3 passes out the distal end 1522 into the sleeve 1530 and then from there into the proximal passageway 1551. In other examples, the catheter 1520 may be positioned differently and may be attached to a left side 1535 of the sleeve 1530, or indeed to a front or rear side. Furthermore, the distal end 1522 of the catheter 1520 may be blocked and an outlet (not shown) may be provided in a lateral side of the catheter 1520 to allow fluid to pass out into the sleeve 1530.

Referring to FIG. 10, in a third example fluid passes from the sleeve 1530 into the catheter 1520 and then into the proximal passageway 1551. The configuration of this example is similar to the first example shown in FIG. 8 and so only the differences are described. In this example, the proximal passageway 1551 is sealed between the left side 1551b and right side 1551a such that the catheter 1520 is sealed on all sides. As such, fluid may only pass from the sleeve 1530/catheter 1520 into the proximal passageway 1551 if it is within the catheter 1520. The left lateral side 1524 of the catheter 1520 comprises an oval sleeve intake opening 1525 adjacent to the proximal passageway 1551 which permits the passage of fluid travelling within the sleeve 3 into the catheter to join fluid travelling within the catheter 2. From here, the fluid 2, 3 passes out the distal end 1522 of the catheter 1520 and into the proximal passageway 1551. Many other configurations may be possible to achieve the same effect, for example, the sleeve 1530 may be attached to the outer sides of the catheter 1520 itself.

Referring to FIGS. 11 to 13, a variation on the valve assembly 1550 described above is shown. In this example, the valve assembly 1550 is positioned in-line between two sections of tubing rather than directly connected to a fluid collection bag as with the first embodiment. This could be advantageous to enable more compact or flexible packaging of the catheter assembly, or to position the valve assembly 1550 in a more convenient place for the user to interact with.

In this example, the valve assembly 1550 is configured such that in the second configuration fluid flow is permitted in both directions, that is from the catheter 1520 towards the fluid collection bag 1510 and vice versa. This retains the core function of the valve assembly 1550 to selectively allow the fluid collection bag 1510 to be drained by switching from the first to second configuration, but does so with a slightly simplified design as one-way flow is not required in the second configuration.

In this example, the valve body could therefore be a single check valve arranged to prevent fluid flow from the fluid collection bag towards the catheter. The button 1554 may be configured to inhibit the check valve from closing when the valve assembly 1550 is in the second configuration thus enabling two-way flow.

Referring to FIGS. 8 to 11, a second embodiment of a packaged catheter assembly 1100 shares many of the features of the first embodiment described above and like numerals are used to denote like features.

The assembly 1100 comprises a fluid collection bag 1110, a catheter 1120, a sleeve 1130, a fluid reservoir (not shown, but which may be identical to that in the first embodiment) and a pouch 1170 containing the fluid collection bag 1110, catheter 1120, sleeve 1130 and fluid reservoir. The catheter 1120 has a proximal end 1121 for insertion into the body and a distal end 1122, and the fluid collection bag 1110 is arranged to receive fluid from the distal end 1122 of the catheter 1120.

In this embodiment, the catheter 1120 is a male urinary catheter made from a hydrophilic thermoplastic elastomer (TPE). Obviously those skilled in the art will be able to select suitable alternative examples.

The pouch 1170 is formed from a front wall 1171a and a rear wall 1171b of identical shape and size, two side walls 1171c, and a peripheral seal joining the periphery of the walls to form the pouch 1170 and containing its contents (i.e. the closed catheter assembly and fluid reservoir 1140).

The peripheral seal defines a base 1172a, a left lateral edge 172b, a right lateral edge 1172c and an upper edge 1172d of the pouch 1170. The left lateral edge 1172b and right lateral edge 1172c being defined as the left and right sides of the pouch 1700 when viewing the pouch 1170 with the rear wall 1171b behind the front wall 1171a, the base 1172a at the bottom of the pouch 1170 and the upper edge 1172d at the top of the pouch 1170. The peripheral seal thus defines a pouch 1170 that is generally rectangular and suitably has a width between the lateral edges of between 60-110 mm, preferably 90-110 mm, for example about 100 mm, and a height from the base 1172a to the upper edge 1172d of between 100 to 250 mm, preferably 120 to 140 mm, for example about 130 mm. The base 1172a defines the bottom of the pouch 1170 in use, and the upper edge 1172d the top. However, in other embodiments alternative shapes and sizes could be conceived, such as elliptical or circular walls forming correspondingly shaped pouches, or even a tubular pouch or pouch with more than two walls, e.g. a prism or other polygon.

The peripheral seal may be formed from any suitable bonding/sealing methods such as chemical adhesives, chemical bonding, ultrasonic bonding, or mechanical bonding. In alternative embodiments, the pouch 1170 may be formed from a single piece of material. The pouch may then be formed by folding this single piece of material along a pouch fold line to form the walls. In addition, in some embodiments any number of folds may be used to form the pouch 1170. For example, none of the edges may comprise a fold, one edge may comprise a fold (as in the illustrated example), or two or more of the edges may comprise a fold.

In this embodiment, the side walls 1171c are elongate in a direction parallel to the length of the pouch 1170 and extend between the front and rear walls 1171a, 1171b along the left and right lateral edges 1172b, 1172c of the pouch 1170. Along the base 1171a and upper edge 1172d of the pouch 1170, the front and rear walls 1171a, 1171b are sealed to each other directly. As such, in this embodiment, the side walls 1171c allow the front and rear walls 1171a, 1171d to separate to increase the internal volume of the pouch 1170.

In this embodiment, the pouch 1170 is formed from an opaque material and as such provides a discrete package for its contents which can make the user more comfortable in carrying the packaged catheter assembly 1100 in day-to-day life. In addition, the exterior of the pouch 1170 may have a textured or printed appearance, for example a matte appearance, and feel to reduce the impression that it is a medical device.

In this embodiment, the pouch 1170 also comprises an interaction region which forms the majority of the front wall 1171a of the pouch 1170. In this embodiment, the interaction region comprises a break-away region in the form of a rectangular peel-away region 1174. The peel-away region 1174 comprises a base 1174a, left lateral edge 1174b, right lateral edge 1174c and upper edge 1174d that correspond to the base 1172a, left lateral edge 1172b, right lateral edge 1172c and upper edge 1172d of the pouch 1170 respectively. Consequently, the peel-away region 1174 comprises a width between its left lateral edge 1174b and right lateral edge 1174c and a height between its base 1174a and upper edge 1174d. The width of the peel-away region 1174 is between 50-100%, 70-90% or most preferably 80-90% of the width of the pouch 1170, for example 85% of the width of the pouch 1170. The height of the peel-away region 1174 is between 50-100%, 60-90% or most preferably 70-80% of the height of the pouch 1170, for example 75% of the height of the pouch 1170. In this embodiment, the peel-away region 1174 is positioned centrally with respect to the width and height of the pouch 1170.

In this embodiment, the corners of the peel-away region 1174 are rounded to provide a smoother and less jagged appearance to the peel-away region 1174. In other embodiments, the corners may not be rounded.

In other embodiments, the break-away region may have a different shape or size as required by the shape/size of the pouch 1170 or the contents of the pouch 1170. It may also not be positioned centrally with respect to the width and/or height of the pouch 1170, for example it may be positioned closer to one edge of the pouch than another.

In this embodiment, the peel-away region 1174 is formed from the same material as the front wall 1171a of the pouch 1170 and is also the same colour to the front wall 1171a, this gives the pouch 1170 a more discreet appearance. In other embodiments, the peel-away region 1174 may be formed of a different material and/or a different colour/shade of colour.

In this embodiment, the front wall 1171a of the pouch 1170 comprises an access aperture 1178a in a position corresponding to the peel-away region 1174. The peel-away region 1174 is attached to the front wall 1171a of the pouch 1170 by a frangible adhesive positioned on the front wall 1171a beneath the base 1174a of the peel-away region 1174. The adhesive 1178b is spread over a rectangular adhesive region 1178b of the front wall 1171a between the access aperture 1178a and base 1172a of the pouch 1170. The adhesive region 1178b has an aspect ratio (length:width) of 1:3 and has a width approximately 50% of the width of the pouch. The adhesive region 1178b is positioned centrally across the width of the pouch 1170 and is completely covered by the peel-away region 1174 when the pouch 1170 is closed. This allows the user to form an opening in the pouch 1170 by peeling the peel-away region 1174 away from the front wall 1171a and breaking the frangible adhesive.

In other embodiments the break-away region may be attached to the front wall 1171a by any suitable bonding/sealing method, for example chemical adhesives, chemical bonding, ultrasonic bonding, or mechanical bonding. In other (tear-away) embodiments, a line of weakness or tear-line in the front wall may define the tear-away region and allow the user to form an opening in the pouch by separating the tear-away region from the front wall.

In this embodiment, the peel-away region 1174 is formed from the same piece of material as the front wall 1171a. Consequently, the upper edge 1174d of the peel-away region 1174 is both part of the peel-away region 1174 and part of the front wall 1171a of the pouch 1170 and defines a fold-line about which the peel-away region 1174 may pivot to allow access to the pouch 1170.

In this embodiment, the upper edge 1174d of the peel-away region 1174 is consequently configured not to separate from the front wall 1171a, this prevents the peel-away region 1174 from separating from the pouch 1170 and reduces the number of items that the user must worry about during use of the catheter assembly 1100. In other embodiments, the peel-away region 1174 may be configured to fully separate from the pouch 1170—for example to allow for easier access to the contents.

In this embodiment, the peel-away region 1174 comprises a front opening tab 1175. The front opening tab 1175 projects from the base 1174a of the peel-away region 1174.

The base 1174a of the peel-away region 1174 comprises straight edges that are slanted towards the centre of the base 1174a. The front opening tab 1175a is positioned at the centre of the base 1174a and spans approximately 50% of the width of the base 1174a. The front opening tab 1175 has a length from the base 1174a to a tip of the front opening tab 1175a that is approximately equal to its width, with a semi-circular end defining the tip.

In this embodiment, the front opening tab 1175 is not adhered to the front wall 1171a of the pouch 1170. The front opening tab 1175 is spaced from the front wall 1171a due to the curvature in the front wall 1171a as it bends to meet the rear wall 1171b at the base 1172a of the pouch 1170. This makes the front opening tab 1175 easier for a user to grasp and open the pouch 1170.

In this embodiment, the base 1172a of the pouch 1170 extends beyond the lateral edges of the front tab 1175 as the front tab 1175 does not extend across the full width of the pouch 1170. This makes it easier to separate the tab 1175 from the pouch 1170.

In this embodiment, the front opening tab 1175 comprises an opening marker 1177. The opening marker 1177 comprises a region of the tab 1175 that comprises a logo, marking, a different colour/shade of colour, or different texture/tactile feel than the rest of the tab 1175. In this embodiment, the opening marker 1177 comprises a triangular marker. The marker 1177 is an equilateral triangle positioned centrally on the tab 1177 and pointing towards the centre of the peel-away region 1174. Thus, the marker 1177 directs the user to pull the front opening tab 1175 away from the base 1172a of the pouch 1170 to open the pouch 1170. In other embodiments, a different logo/shape/marker could of course be used to achieve the same effect. In addition, a gripping hole or other tactile element could be used to help the user grasp the tab 1175 more easily.

In this embodiment, the pouch 1170 comprises a first compartment 1181 and a second compartment 1182 separated by an internal wall 1183. The internal wall 1183 comprises a fluid impermeable material that is the same as the material of the front wall 1171a and rear wall 1171b, but, in this embodiment the internal wall 1183 is transparent/translucent to allow the user to see the contents of the first compartment 1181 through the wall 1183. The internal wall 1183 spans the entire width of the pouch 1170 from the left lateral edge 1172b to the right lateral edge 1172c. The internal wall 1183 spans from the base 1172a of the pouch 1170 up approximately 50-90% of the height of the pouch 1170 and most preferably 60% of the height of the pouch 1170. Consequently, an upper (separating) edge 1184 of the internal wall 1183 is revealed when the peel-away region 1174 is at least partially removed from the pouch 1170. The internal wall 1183 is sealed to the pouch 1170 by the peripheral seal which, in this embodiment, seals together the front wall 1171a, internal wall 1183 and rear wall 1171b.

In this embodiment, the internal wall 1183 comprises a cut-away region 1185 cut into the upper edge 1184 of the internal wall 1183. The cut-away region 1185 is centrally located across the width of the pouch 1170 and spans approximately 30% of the width of the peel-away region 1174. The cut-away region 1185 has a semi-circular shape. Of course, in other embodiments the cut-away region 1185 may have any suitable shape, such as rectangular, triangular, ellipsoidal or irregularly shaped.

In this embodiment, the upper edge 1184 is sealed to the rear wall 1171b of the pouch 1170 along its entire length except at the cut-away region 1185. This helps to ensure that the contents of each compartment 1181, 1182 does not inadvertently leave each compartment 1181, 1182. The cut-away region 1185 provides access between the first compartment 1181 and second compartment 1182 as the internal wall 1183 is not sealed to either the first wall 1171a or second wall 1171b at the cut-away region 1185.

In this embodiment, the first compartment is defined by the rear wall 1171b and internal wall 1183, and the second compartment is defined by: the internal wall 1183 and the front wall 1171a at locations below the upper edge 1184 of the internal wall 1183; and the rear wall 1171b and front wall 1171a above the upper edge 1184 of the internal wall 1183. Thus, the first and second compartments 1181, 1182 comprise two parallel and planar compartments with access provided between them at the cut-away region 1185 of the internal wall 1183.

In this embodiment, the fluid reservoir is configured to retain a wetting fluid and release wetting fluid to activate the surface of the catheter 1120. In this embodiment, the wetting fluid is water and interacts with the hydrophilic surface of the catheter 1120 to render it lubricious. In other embodiments, other wetting fluids may be used and they may be polar (e.g. water-based) or non-polar (e.g. oil-based) depending on the catheter's surface properties.

In this embodiment, the fluid reservoir comprises a burstable rectangular sachet positioned within the first compartment 1181 of the pouch 1170 adjacent to the base 1172a of the pouch 1170. The user can release wetting fluid held by the fluid reservoir by compressing the fluid reservoir, for example by pinching it between their fingers.

The user can advantageously do this while the fluid reservoir is contained within the pouch 1170 as described in more detail below. To facilitate the user in activating the fluid reservoir, the pouch 1170 comprises a wetting marker 1177 which in this embodiment is the same feature as the opening marker 1177 described above. The wetting marker 1177 comprises a region of the pouch 1170 that is a different colour or texture to the rest of the pouch 1170 to allow the user to easily identify the position of the fluid reservoir and where to press to activate it and release wetting fluid into the pouch 1170. In this embodiment, the wetting marker 1177 is disposed on the peel-away region 1174. In other embodiments, the fluid reservoir may be configured to release wetting fluid through a different means, for example by twisting or bursting the fluid reservoir in a different way.

In this embodiment, the fluid collection bag 1110 comprises a front panel 1111a, a rear panel 1111b of identical shape and size and a base panel 1111d that spans between the front panel 1111a and rear panel 1111b. A peripheral bond joins the peripheries of the panels to form the bag 1110. The peripheral bond defines a base 1112, a right lateral edge 1113, a left lateral edge 1114 and an upper edge 1115. The right lateral edge 1113 and left lateral edge 1114 being defined as the right and left sides of the bag 1110 when viewing the bag 1110 with the rear panel 1111b behind the front panel 1111, the base 1112 at the bottom of the bag 1110 and the upper edge 1115 at the top of the bag 1110. The peripheral bond thus defines a bag 1110 that is generally rectangular and suitably has a width between the lateral edges of between 10 and 20 cm, and most preferably between 12 and 15 cm, and a height from the base 1112 to the upper edge 1115 of between 25 and 40 cm, 30 and 35 cm, or most preferably 33 and 35 cm. The bag is thus configured to hold at least 700 ml of fluid released from the user through the catheter safely. The base 1112 defines the bottom of the bag 1110 in use, and the upper edge 1115 the top. However, in other embodiments alternative shapes and sizes could be conceived, such as elliptical or circular panels forming correspondingly shaped bags, or even a tubular bag or bag with more than two panels, e.g. a prism or other polygon.

Notably, in this embodiment, the fluid collection bag 1110 comprises a base panel 1111d that spans between the front panel 1111a and rear panel 1111b along the base 1112 of the peripheral bond. The base panel 1111d is lens shaped in that it has two curved edges bounding a convex region. The curved edges are joined to the base edges of the front and rear panels 1111a, 1111b respectively and the vertices where the curved edges meet are at the left and right lateral edges 1113, 1114 of the fluid collection bag 1110. The base panel 1111d allows the front panel 1111a and rear panel 1111b to separate along the base 1112.

Consequently, the base panel 1111d allows the fluid collection bag 1110 to have a greater internal volume and also to be free-standing when partially filled with fluid as described below.

The base 1112 is formed by two flanges 1112A, 1112B; the rear panel joined to the base panel by the peripheral bond forms a first flange 1112A and the front panel joined to the base panel by the peripheral bond forms the second flange 1112B. The two flanges 1112A, 1112B extend from the two curved edges where the base panel 1111d meets the rear panel 1111b and the front panel 1111a respectively.

In this embodiment, the front, rear and base panels 1111a, 1111b, 1111d comprise a flexible plastics material, for example polypropylene (PP), polyethylene terephthalate (PET), low density polyethylene (LDPE), metalized polyester (MET PET) orientated polypropylene (OPP), polyvinylpyrrolidone (PVP), or polyvinyl chloride (PVC).

In this embodiment, the upper edge 1115 of the bag 1110 is tapered towards a neck 1150 which facilitates the fluid connection between the fluid collection bag 1110 and the catheter 1120. The neck 1150 is positioned adjacent to the right lateral edge 1113 of the fluid collection bag 1110 and spans the right hand third of the upper edge 1115. The neck 1150 has an aspect ratio (length:width) of 2:1 at its base and is tapered such that its width is reduced by approximately 20-50% at its tip, which is flat with rounded corners.

In this embodiment, the neck 1150 comprises an inlet 1153 positioned centrally on the neck 1150 and in the front panel 1111a of the bag 1110. The inlet 1153 is configured to receive the catheter 1120 and comprises an aperture through which the catheter 1120 projects and in which the catheter 1120 is retained.

In this embodiment, the fluid collection bag 1110 is a closed bag that is not intended to release fluid contained within it. However, in other embodiments, the fluid collection bag 1110 may comprise an outlet to allow fluid to be released from the bag 1110.

In this embodiment, the fluid collection bag 1110 also comprises a retaining element in the form of a strap 1117. The strap 1117 is configured to retain the fluid collection bag 1110 in a stowed (rolled) configuration before use. The strap 1117 comprises a strip of material configured to extend all the way around the rolled bag 1110 and seal to itself, for example through a chemical adhesive, hook-and-hook, or hook-and-loop fasteners. In this embodiment, hook-and-hook type fasteners are used to allow re-sealable adhesion to a variety of surfaces/objects as described further below.

Alternatively, a re-sealable adhesive such as an epoxy resin or alternatively a silicone-based adhesive may be used. In addition, one end of the strap 1117 is permanently adhered to the front panel 1111a of the fluid collection bag 1110 for example through a weld, to ensure the strap 1117 does not become fully detached from the bag 1110 and is easier to use.

In this embodiment, the strap 1117 is also configured to releasably attach the rolled fluid collection bag 1110 to the underside of the peel-away region 1174. Consequently, when the pouch 1170 is opened, the fluid collection bag 1110 is pulled through the access aperture 1178a as it remains on the underside of the peel-away region 1174 until the user removes it from the pouch 1170 as described below.

In this embodiment, the strap 1117 is also configured to retain the catheter 1120 in a stowed (folded) configuration after use. The catheter 1120 may be coiled and then sealed against the bag 1110 using the strap 1117. The upper region of the bag 1110 adjacent to the upper edge 1115 may be folded (with the front panel 1111a on the inside of the fold) and the strap 1117 attached to (the rear panel 1111b of) the bag 1110 to retain the bag 1110 in the folded configuration with the catheter 1020 stowed against it. Thus, the bag 1110 and catheter 1120 are more convenient to carry and dispose of after use.

In this embodiment, the strap 1117 comprises a release mechanism in the form of a pull-tab 1118 which is a short section of the strap 1117 that is configured to be pulled by the user to release the strap 1117. In this embodiment, the pull-tab 1118 is the end of the strap 1117 distal from the end permanently attached to the fluid collection bag 1110.

In this embodiment, the pull-tab 1118 comprises a release marker in the form of a pull-marker 1119. The pull-marker 1119 is a region of the pull-tab 1178 that comprises a logo, marking, a different colour/shade of colour, or different texture/tactile feel than the rest of the tab 1178. In this embodiment, the pull-marker 1119 comprises a triangular marker. The marker 1119 is an equilateral triangle positioned centrally on the tab 1119 and pointing towards the opposite end of the strap 1117. Thus, the marker 1119 directs the user to pull the tab 1118 to release the strap 1117. In other embodiments, a different logo/shape/marker could of course be used to achieve the same effect. Of course, the marker 1119 may be recognisable both visually and by touch. In addition, a gripping hole or other tactile element could be used to help the user grasp the tab 1118 more easily.

In this embodiment, the distal end 1122 of the catheter 1120 comprises a funnel 1123. The funnel 1123 is positioned within the fluid collection bag 1110 and comprises a relatively rigid material, such as HDPE, compared to the fluid collection bag 1110. The funnel 1123 is sized such that it cannot pass through the inlet 1153 and as such, is configured to prevent removal of the catheter 1120 from the fluid collection bag 1110.

In this embodiment, the fluid collection bag 1110 is provided in a stowed folded configuration and in a different compartment as the fluid reservoir, consequently, the fluid collection bag 1110 is provided in the second compartment 1182 attached to the peel-away region 1174 as mentioned above. The inlet 1153 is positioned closer to the cut-away region 1185 and upper edge 1184 of the internal wall 1183 than the rest of the stowed fluid collection bag 1110 and the catheter 1120 extends from the inlet 1153 between the upper edge 1184 of the internal wall 1183 and upper edge 1172d of the pouch 1170 into the first compartment 1181.

The position and configuration of the catheter 1120 in the first compartment 1181 is not particularly limited so long as it is not curved to the extent that damage to the catheter is likely, for example from kinking. In this embodiment, the catheter 1120 is arranged within the first compartment 1181 in a curved/coiled configuration, this helps ensure that the catheter 1120 is not excessively curved and minimises risk of damage.

In this embodiment, the sleeve 1130 comprises two handling elements 1131. Each handling element 1131 comprises a tubular sheet material through which the catheter 1120 extends and which allows the handling element 1131 to be moved along the length of the catheter 1120. Each handling element 1131 is approximately 2-8 cm in length, preferably 3-7 cm, for example 5 cm in length. Consequently, each handling element 1131 and both handling elements 1131 together span a minority of the length of the catheter 1120. This leaves the majority of the catheter 1120 uncovered and as such easily wetted by wetting fluid released from the fluid reservoir as described below.

In this embodiment, the sleeve 1130 comprises a line of weakness 1132 that may be broken to form the two handling elements 1132. This simplifies manufacture of the catheter assembly and can allow the handling elements 1131 to more securely remain in a given position prior to use. In this embodiment, the line of weakness 1132 comprises a line of perforations around the circumference of the sleeve 1130. In other embodiments, the handling elements 1132 may be independent but sealed together by a temporary seal present between them, for example a tack weld. In other embodiments, each handling element 1131 may be independent of the other and not adhered or sealed to the other.

While in this embodiment the sleeve 1130 comprises two handling elements 1131, in other embodiments, the sleeve 1130 may comprise a single handling element 1131 or more than two handling elements 1131 as required.

In this embodiment, one handling elements 1131 is located in a position corresponding to the cut-away region 1185 such that its position is restricted and it is easily accessible to the user when opening the pouch 1170. In this position, the handline element 1131 also helps to prevent liquid from passing through the cut-away region 1185 and into the second compartment 1182. As described above, the fluid reservoir is arranged in the first compartment 1181 and the catheter 1120 extends from the fluid collection bag 1110 into the first compartment 1181.

Prior to use, the pouch 1170 is provided sealed closed with the contents arranged within it as mentioned above. To ensure the catheter 1120 is wetted prior to use, the user must activate the fluid reservoir by locating it within the pouch 1170 and squeezing it to rupture the fluid reservoir and release wetting fluid into the first compartment 1181. This can be done with the aid of the wetting marker 1177 on the outside of the pouch 1170.

Advantageously, once wetting fluid is released into the first compartment 1181 it is prevented from reaching the fluid collection bag 1110 and sleeve 1130 by the internal wall 1183. This ensures that the wetting fluid is most efficiently used to wet the catheter 1120, the majority of which is in the first compartment including the proximal end 1121 of the catheter. In addition, it ensures that the fluid collection bag 1110 and sleeve 1130 remain dry and easier to handle.

Once the catheter 1120 has been wetted, the contents of the pouch 1170 can be accessed by opening the pouch 1170. The pouch 1170 is opened by separating the opening tab 1175 from the pouch 1170 to break the adhesive at the adhesive region 1178b. As the tab 1175 and pouch 1170 are separated, the peel-away region 1174 peels away from the front wall 1171a of the pouch 1170 revealing the contents of the second compartment 1182.

Once the pouch 1170 has been opened, which in some embodiments may involve completely removing the break-away region from the pouch 1170, the fluid collection bag 1110, catheter 1120 and sleeve 1130 may be removed from the pouch 1170. Preferably, the user grasps the dry folded stowed fluid collection bag 1110, for example using the pull-tab 1118, and/or one or both dry handling elements 1131 of the sleeve 1130 and uses them to pull the now wetted catheter out of the pouch 1170 via the second compartment 1182.

Before use, the fluid collection bag 1110 must be unrolled to ensure that the collection of liquid is possible. This can be done by pulling the pull-tab 1118 in the direction of the pull-marker 1119 with respect to the rest of the bag 1110 to release the strap 1117 and unroll the bag 1110. The handling elements 1131 can be used to handle the catheter 1120 and progressively introduce it into the body until urine flows therethrough and into the fluid collection bag 1110.

In this embodiment, the base panel 1111d of the bag 1110 is initially provided in a stowed configuration in which it is folded up between the front and rear panels 1111a, 1111b. As liquid 1101 enters the bag 1110, the front and rear panels 1111a, 1111b may be separated and the base panel 111d unfolded such that it is flat and substantially perpendicular to the front and rear panels 1111a, 1111b. Liquid 1101 inside the bag 1110 collects at the base panel 1111d and consequently, the bag 1110 is able to stand up while it is filled. This provides additional convenience to the user as they need not hold or hand the bag during use.

Once the user has emptied their bladder, the catheter 1120 may be removed from the body, for example using the sleeve 1130/handling elements 1131. The catheter 1120 may then be folded/coiled and stowed against the fluid collection bag 1110 using the strap 1117. As mentioned above, the fluid collection bag 1110 may be folded close to the upper edge 1115 and the folded/coiled catheter 1120 collected adjacent to the neck 1150. The strap 1117 is then used to secure the fluid collection bag 1110 and catheter 1120 in this position by attaching it to the fluid collection bag 1110, for example on the rear panel 1111b. The fluid collection bag 1110 and catheter 1120 are then in a conveniently arranged for disposal or storage until disposal.

While the embodiment disclosed above may be described as a closed catheter assembly in that fluid is collected in a fluid collection bag, other embodiments may not comprise a fluid collection bag and may be open catheter assemblies in that fluid is not collected during use.

The one or more embodiments are described above by way of example only. Many variations are possible without departing from the scope of protection afforded by the appended claims.

1. A catheter assembly comprising a fluid collection bag and a catheter; the fluid collection bag comprising two panels, a peripheral bond between the two panels, and an inlet to receive fluid from the catheter, wherein: the two panels are configured to form a base of the fluid collection bag distal from the inlet; and the fluid collection bag is configured to stand up on the base.

2. The catheter assembly of claim 1 wherein the catheter is a male urinary catheter.

3. The catheter assembly of any preceding claim wherein the fluid collection bag further comprises a base panel configured to form the base of the fluid collection bag.

4. The catheter assembly of claim 3 wherein the fluid collection bag comprises a front panel, a rear panel and the base panel, and the front and rear panels are joined to opposite sides of the base panel.

5. The catheter assembly of claim 4 wherein the front panel and rear panel are joined by the peripheral bond along an upper edge, a left lateral edge and a right lateral edge of the fluid collection bag.

6. The catheter assembly of claim 4 wherein the fluid collection bag further comprises two side panels, the front and rear panels are joined to opposite sides of each side panel, and the base panel is joined to one side of each side panel.

7. The catheter assembly of claim 6 wherein the base panel is rectangular and each side panel is triangular.

8. The catheter assembly of claim 6 or claim 7 wherein the front panel and rear panel are joined by the peripheral bond along an upper edge of the fluid collection bag.

9. The catheter assembly of any of claims 6 to 8 wherein the two side panels are configured to allow the front and rear panels to separate along a left lateral edge and right lateral edge of the fluid collection bag.

10. The catheter assembly of any of claims 6 to 9 wherein the fluid collection bag is wedge-shaped.

11. The catheter assembly of any of claims 3 to 10 wherein the base panel and/or the side panels comprise a stowed configuration in which the base panel/side panels are configured to reduce the internal volume of the fluid collection bag.

12. The catheter assembly of claim 11 wherein the base panel/side panels are folded in the stowed configuration.

13. The catheter assembly of claim 12 wherein the base panel/side panels are configured to unfold as liquid enters the fluid collection bag.

14. The catheter assembly of any preceding claim wherein the catheter comprises a funnel configured to engage the inlet of the fluid collection bag.

15. The catheter assembly of any preceding claim wherein the fluid collection bag comprises a fill level indicator configured to allow the user to visualise fluid within the bag and the fill level indicator is positioned adjacent to the inlet.

16. A method of manufacturing a catheter assembly comprising the steps of providing a catheter having a proximal end for insertion into the body and a distal end and forming a fluid collection bag, the fluid collection bag being formed by providing two panels, creating a peripheral bond between the two panels, and providing an inlet in the fluid collection bag to receive fluid from the catheter, wherein: the two panels are configured to form a base of the fluid collection bag distal from the inlet; and the fluid collection bag is configured to stand up on the base.

17. The method of claim 16 further comprising providing a funnel at the distal end of the catheter and positioning the funnel inside the fluid collection bag.

18. A method of preparing a catheter assembly for use, the catheter assembly comprising a catheter and the fluid collection bag, the fluid collection bag comprising two panels, a peripheral bond between the two panels, and an inlet to receive fluid from the catheter, the method comprising using the two panels to form a base of the fluid collection bag distal from the inlet; and standing the fluid collection bag up on the base.

19. The method of any of claims 16 to 18 wherein the catheter assembly is the catheter assembly of any of claims 1 to 15.
